(19) United States
(12) Patent Application Publication
(10) Pub. No.: US 2024/0415809 A1
(43) Pub. Date: Dec. 19, 2024

(54) PHARMACEUTICAL COMBINATIONS

(71) Applicant: ZYDUS LIFESCIENCES LIMITED, Ahmedabad, Gandhinagar, Gujarat (IN)

(72) Inventors: Mukul JAIN, Ahmedabad, Gandhinagar, Gujarat (IN); Suresh GIRI, Ahmedabad, Gandhinagar, Gujarat (IN); Anish SHARMA, Ahmedabad, Gandhinagar, Gujarat (IN); Kannan Essakimuthu MUTHAIYYAN, Ahmedabad, Gandhinagar, Gujarat (IN); Pranav Dhirajbhai JOGANI, Ahmedabad, Gandhinagar, Gujarat (IN); Vinay Satishchandra UPADHYAY, Ahmedabad, Gandhinagar, Gujarat (IN); Nakul Pitambarbhai PATEL, Ahmedabad, Gandhinagar, Gujarat (IN)

(21) Appl. No.: 18/701,272

(22) PCT Filed: Oct. 18, 2022
(86) PCT No.: PCT/IB2022/059976

§ 371 Date: Apr. 15, 2024

(30) Foreign Application Priority Data
Oct. 19, 2021 (IN) 202121047338

(51) Int. Cl. A61K 31/4035; A61K 31/437; A61K 31/56; A61K 31/606; A61K 45/06

(52) U.S. Cl. CPC A61K 31/4035; A61K 31/437; A61K 31/56; A61K 31/606; A61K 45/06

(57) ABSTRACT

Present invention relates to combination of Apremilast or pharmaceutically acceptable salts thereof and Rifaximin or its polymorphs or isomers or pharmaceutically acceptable salts. Present invention provides the pharmaceutical composition of Apremilast or pharmaceutically acceptable salts thereof and Rifaximin or its polymorphs or isomers or pharmaceutically acceptable salts. Present invention also provides use of the said combination or pharmaceutical composition for the treatment of inflammatory bowel disease.

### FIELD OF THE INVENTION

Present invention relates to combination of Apremilast or pharmaceutically acceptable salts thereof and Rifaximin or its polymorphs or isomers or pharmaceutically acceptable salts. Present invention provides the pharmaceutical composition of Apremilast or pharmaceutically acceptable salts thereof and Rifaximin or its polymorphs or isomers or pharmaceutically acceptable salts. Present invention also provides use of the said combination or pharmaceutical composition for the treatment of inflammatory bowel disease.

### BACKGROUND OF THE INVENTION

Inflammatory bowel disease (IBD) mostly affects the gastrointestinal tract by chronic inflammation. It is chronic as well as relapsing and refractory inflammatory condition that results from the interactions of gene susceptibility, environmental factors, disturbance of immune homeostasis, and sometimes microbiological anomaly in the gastrointestinal tract, yet the exact cause of inflammatory bowel disease is unknown but most of the references directs that cause of IBD is a result of disturbed immune system and most of the time it triggered from bacterial or viral infection. (Gastroenterol Clin North Am. 2017 December; 46 (4): 769-782.) IBD has most common symptoms of diarrhea, abdominal pain, rectal bleeding or stool in blood, weight loss or fatigue. Inflammatory bowel disease known for its two sub types namely Crohn's disease and Ulcerative colitis. As per the reports Ulcerative colitis is a global health challenge with a prevalence of over 0.3% worldwide (Br J Pharmacol. 2019; 176:2209-2226).

Ulcerative colitis (UC) in particular, is a chronic inflammatory condition of large intestine. There are many reasons recognized for cause of UC. Among them, gut microbiota is recognized as an important factor involved in promoting and/or maintaining the inflammatory process typical for IBD. The concentration of intestinal bacteria in IBD patients is higher than normal, gradually increasing with the severity of the disease. A breakdown in the qualitative balance between protective and harmful bacteria (dysbiosis) has also been proposed as a potential mechanism. Indeed, in Crohn's disease, an increased presence of Campylobacter concisus and E. coli as well as a substantial decrease in the amount of the anti-inflammatory commensal Faecalibacterium prausnitzii, has been reported. On the other hand, it has been suggested that Fusobacterium varium can promote the development of ulcerative colitis. (World J Gastroenterol 2011 Nov. 14; 17 (42): 4643-4646).

Currently there are few drug therapy or surgery available as a treatment. Current drug therapy includes very few therapies non-biologics therapy (such as mesalamine, corticosteroids, immuno-suppressants), biologics therapy (such as infliximab, adalimumab, golimumab, vedolizumab) and oral immunomodulatory agent (such as tofacitinib) for the treatment of UC. However, many patients do not respond to or are intolerant of available therapies. Biologic and oral immunomodulatory treatments are associated with adverse events (AEs) such as serious infections and malignancies. Thus, there are still unmet need for improved, well-tolerated and safer therapy for IBD and specifically for UC. Other than mentioned, Rifaximin is an anti-bacterial drug that should be used to treat or prevent infections caused by bacteria. It has been approved for treating travelers' diarrhea, hepatic encephalopathy and irritable bowel syndrome with diarrhea. Rifaximin has also shown positive effects for controlling gut microorganisms. However only higher doses of Rifaximin 550 mg thrice a day (up to 1650 mg total dose) shows results in inflammatory bowel disease.

WO 2005044823 and WO 2006094662 discloses various polymorphic forms of Rifaximin. WO 2006094737 discloses gastro-resistant formulation of Rifaximin. Further U.S. Pat. No. 9,988,398B2 disclosed polymorphic Form Z of Rifaximin characterized by X-ray powder diffraction having characteristic peaks expressed in degrees 2θ (±0.2° 2θ) at about 5.1°, 7.1°, 8.3°, and 8.6°±0.2 2θ.

In past few years, immuno-suppressants, corticosteroids, phosphodiesterase-4 inhibitors and few biological drugs have shown therapeutic efficacies for the treatment of UC symptoms (Clinical Gastroenterology and Hepatology 2020; 18:2526-2534).

Apremilast is PDE4 inhibitor, has been approved for the treatment of oral ulcers associated with Behcet's syndrome, Plaque psoriasis and Psoriatic arthritis. The recommended dose of Apremilast is 30 mg dose twice daily. Apremilast is an oral small molecule, acts intracellularly to modulate inflammatory mediators (Biochem Pharmacol 2012; 83:1583-1590.) The important enzyme PDE4 regulates inflammatory response by increasing production of pro-inflammatory mediators (i.e., tumor necrosis factor a [TNF-a], interleukin [IL]-23) and decreasing production of anti-inflammatory mediators (ie, IL-10) (Biochem Pharmacol 2012; 83:1583-1590.), (Am J. Physiol Lung Cell Mol Physiol 2009; 296: L959-L969.) Apremilast inhibits TNF-α and matrix metalloproteinase 3 production in lamina propria mononuclear cells of patients with inflammatory bowel disease. (J Crohns Colitis 2009; 3:175-182.) Apremilast has been also evaluated for Ulcerative colitis (Clinical Gastroenterology and Hepatology 2020; 18:2526-2534).

U.S. Pat. No. 7,427,638 discloses stereoisomerically pure form of Apremilast. U.S. Pat. No. 7,659,302 discloses method of treating inflammation related disorders such as psoriasis, skin inflammation diseases, atopic dermatitis, contact dermatitis, rheumatoid arthritis, osteoarthritis, systemic lupus erythrematosus, inflammatory bowel disease, Crohn's Disease, Behcet's Disease or colitis.

It has been now surprisingly found that combining Apremilast and Rifaximin shows significant effects on treating/ameliorating inflammatory bowel disease.

### SUMMARY OF THE INVENTION

Present invention relates to the combination of Apremilast or pharmaceutically acceptable salts thereof and Rifaximin or its polymorphs or isomers or pharmaceutically acceptable salts. Present invention also provides the pharmaceutical composition of Apremilast or pharmaceutically acceptable salts thereof and Rifaximin or its polymorphs or isomers or pharmaceutically acceptable salts. This invention also describes the use of said combination or pharmaceutical composition for treatment of inflammatory bowel disease.

### BRIEF DESCRIPTION OF THE FIGURES

FIG. 1: Histological figures representing grades of inflammation (H&E staining) in colon tissue

FIG. 2: Histological figures representing grades of fibrosis (Masson's trichrome staining) in colon tissue

FIG. 3: An illustrative drawing of a pharmaceutical kit comprising two parts divided with perforation. Part A showing a Rifaximin tablet of 500 mg and Part B showing a Apremilast tablet of 15 or 25 mg.

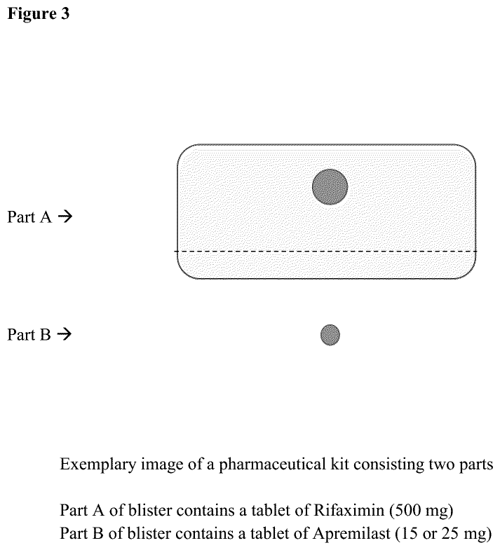

### EMBODIMENTS OF THE PRESENT INVENTION

In an embodiment, present invention relates to combination of Apremilast and Rifaximin.

In another embodiment, present invention relates to combination of stereoisomerically pure Apremilast and Rifaximin.

In yet another embodiment, present invention relates to the combination of Apremilast or stereoisomerically pure Apremilast and the polymorphs of Rifaximin.

In another embodiment, present invention relates to the combination of Apremilast or stereoisomerically pure Apremilast and the polymorphs of Rifaximin wherein polymorphs of Rifaximin are selected from α, β, γ, δ, ε or Z form of Rifaximin.

In another embodiment, present invention relates to a pharmaceutical composition comprising Apremilast, Rifaximin and pharmaceutically acceptable excipients.

In another embodiment, present invention relates to a pharmaceutical composition of Apremilast, Rifaximin is in the form of immediate release composition

In another embodiment, a pharmaceutical composition of Apremilast and Rifaximin is in the form of delay release composition.

In another embodiment, a pharmaceutical composition of Apremilast and Rifaximin is in the form of gastric resistance composition.

In another embodiment, a pharmaceutical kit comprising two parts divided with perforation or a punch or other suitable means having medicaments selected from Apremilast and Rifaximin.

Wherein Apremilast and Rifaximin is provided in suitable therapeutically effective amount of each of them.

In another embodiment, present invention relates to method of preparation of immediate release or gastro resistance or delay release pharmaceutical composition comprising Apremilast and Rifaximin.

In another embodiment, present invention relates to use of combination or pharmaceutical composition comprising Apremilast and Rifaximin for treatment of inflammatory bowel disease.

In another embodiment, present invention relates to method of treating inflammatory bowel disease using a combination or pharmaceutical composition comprising Apremilast and Rifaximin.

In an embodiment the combination and pharmaceutical composition of the present invention, is for use in combination with at least one suitable therapeutic agent.

### DESCRIPTION OF THE INVENTION

Herein, the term “treating” includes abrogating, substantially inhibiting, slowing or reversing the progression of a disease, substantially ameliorating clinical symptoms of a disease or substantially preventing the appearance of clinical symptoms of a disease. The term ‘pharmaceutically acceptable’ relates to its use for both human and animals. The term “excipient” or “pharmaceutically acceptable excipient” refers to pharmacologically inactive substances that are added to a pharmaceutical preparation in addition to the active pharmaceutical ingredient. The term “BID” indicates a dose twice a day. The term “DSS” refers to Dextran Sulfate Sodium. ‘Pharmaceutical Kit’ or ‘Combi kit’ is intended to mean a kit made up of plastic or metallized paper/blister or other suitable ingredients known in the art which is used to keep a drug dosage safe from moisture, sunlight, microbial and dirt contamination.

### Combination of Apremilast and Rifaximin:

As described in above section, present invention relates to combination of Apremilast and Rifaximin.

In an embodiment present invention relates the pharmaceutically combination comprising,

A first component (1) Apremilast or pharmaceutically acceptable salts thereof

A second component (2) Rifaximin or isomers or pharmaceutically acceptable salts.

In another embodiment, present invention relates to combination of stereoisomerically pure Apremilast with Rifaximin. (+)-2-[1-(3-ethoxy-4-methoxyphenyl)-2-methylsulfonylethyl]-4-acetylaminoisoindoline-1,3-dione is known as stereoisomerically pure Apremilast. Wherein stereoisomerically pure Apremilast can be synthesized as per the process described in U.S. Pat. No. 7,427,638.

In another embodiment, present invention relates to combination of stereoisomerically pure Apremilast or a pharmaceutically acceptable salt, solvate or hydrate, thereof and Rifaximin.

In yet another embodiment, present invention relates to the combination of Apremilast or stereoisomerically pure Apremilast and the polymorphs of Rifaximin. Wherein polymorphs of Rifaximin are selected from α, β, γ, δ, ε or Z form of Rifaximin. Polymorphs of Rifaximin are synthesized as per the processes provided in WO2005044823, WO2006094662, U.S. Pat. No. 9,988,398 and other prior arts.

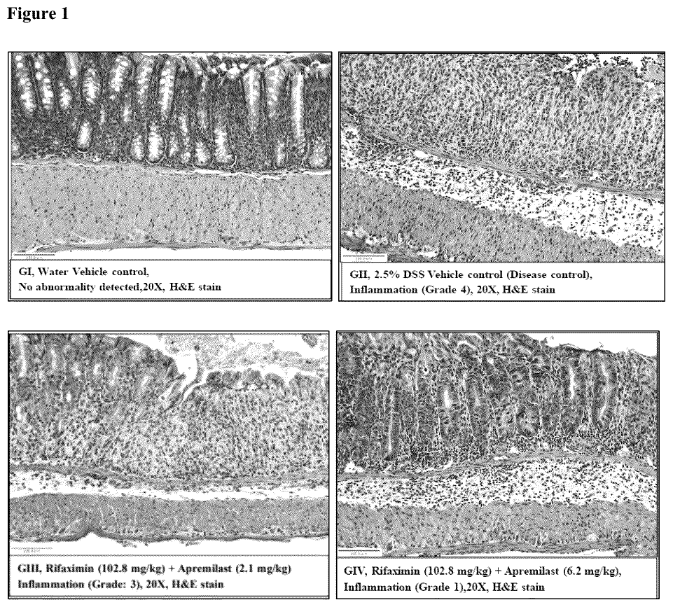

In one of the preferred embodiment, present invention relates to the combination of Apremilast or stereoisomerically pure Apremilast and α-form of Rifaximin. The XRD of α-form of Rifaximin is as provided in FIG. 1 of WO2005044823.

In another preferred embodiment, present invention relates to the combination of Apremilast or stereoisomerically pure Apremilast and β-form of Rifaximin.

In another preferred embodiment, present invention relates to the combination of Apremilast or stereoisomerically pure Apremilast and γ-form of Rifaximin.

In another preferred embodiment, present invention relates to the combination of Apremilast or stereoisomerically pure Apremilast and 8-form of Rifaximin.

In another preferred embodiment, present invention relates to the combination of Apremilast or stereoisomerically pure Apremilast and &-form of Rifaximin.

In another preferred embodiment, present invention relates to the combination of Apremilast or stereoisomerically pure Apremilast and Z-form of Rifaximin. The XRD of Z-form of Rifaximin is as provided in FIG. 1 of U.S. Pat. No. 9,988,398B2.

In another embodiment, present invention relates to combination of Apremilast or stereoisomerically pure Apremilast and the polymorphs of Rifaximin. Wherein Apremilast and Rifaximin is provided in therapeutically effective amount of each of them. Therapeutically effective amount of Apremilast is selected from range of 1.00 mg to 100.00 mg. The preferred amount of Apremilast is selected from range of 10.00 mg to 70.00 mg. The most preferred amount of Apremilast is selected from range of 10.00 mg to 30.00 mg.

Therapeutically effective amount of Rifaximin is selected from range of 1.00 mg to 550.00 mg. The preferred amount of Rifaximin is selected from range of 250.00 mg to 500.00 mg. In more preferred embodiment the amount of Rifaximin is selected from range of 400.00 mg to 500.00 mg. The said therapeutically effective doses of Rifaximin and Apremilast meant to be used for human being in need thereof.

The said combination of Apremilast and Rifaximin is provided to the patients in need thereof by way of oral, parenteral or topical route of administration. In one of the preferred embodiment, the combination is provided to the patients in need thereof by way of oral route or parenteral route of administration. The said combination may give to the patient in need thereof by once a day, twice a day (BID) or thrice a day.

### Pharmaceutical Composition

In another embodiment, present invention relates to a pharmaceutical composition comprising Apremilast, Rifaximin and pharmaceutically acceptable excipients.

In yet another embodiment, present invention relates to a pharmaceutical composition comprising stereoisomerically pure Apremilast, Rifaximin or suitable polymorphs of Rifaximin and pharmaceutically acceptable excipients. Wherein Polymorphs of Rifaximin are selected from α, β, γ, δ, ε or Z forms of Rifaximin.

The pharmaceutical composition of Apremilast, Rifaximin and suitable pharmaceutically acceptable excipients, wherein suitable pharmaceutically acceptable excipients are selected from a disintegrant, coating agent, enteric coating material, a filler, a binder, a glidant, a lubricant and a plasticizer.

Term ‘disintegrants’ are those substances which expand and dissolve when wet, causing the tablet to break apart in the digestive tract, or in specific segments of the digestion process, releasing the active ingredients for absorption. They ensure that when the tablet is in contact with water, it rapidly breaks down into smaller fragments, facilitating dissolution.

Term ‘enteric coating material’ is a material used as a barrier that controls the location of oral medication in the digestive system where it is absorbed. Enteric coating material prevent release of medication before it reaches the specific organ of body. The enteric coating material remain unionize up to certain pH, and therefore remain insoluble.

Term ‘filler’ is intended to mean inert substances used as fillers to create the desired bulk, flow properties, and compression characteristics in the preparation of tablets and capsules.

Term ‘binder’ or ‘binding agent’ is a component that hold the ingredients in a tablet together.

Binders ensure that tablets and granules can be formed with required mechanical strength, and give volume to low active dose tablets.

Term ‘glidant’ is a substance that is added to a powder to improve its flow ability.

Term ‘lubricant’ is a compound that prevent ingredients from clumping together and from sticking to the tablet punches. Common minerals like talc or silica, and fats, e.g. vegetable stearin, magnesium stearate or stearic acid are the most frequently used lubricants in tablets.

Term ‘plasticizer’ refers to additives that decrease the plasticity or viscosity of a material.

In another embodiment of the present invention, is described processes for the preparation of a stable pharmaceutical composition of Apremilast and Rifaximin.

The stable pharmaceutical composition may be made by direct compression, wet granulation or dry granulation methods by techniques known to persons skilled in the art. Thus, for example, in the wet granulation process, the drug is mixed with one or more pharmaceutical excipients and granulated with suitable binding solution to form wet granules, the wet granules are dried and optionally sieved. The dried granules are mixed with one or more suitable excipients from those described elsewhere and then compressed into tablets or filled into capsules.

In direct compression process, the drug is mixed with all the pharmaceutical excipients required and then is either compressed into tablets or filled in capsules.

Term ‘RMG’ refers to Rapid Mixer Granulator which is used for granulation.

Term ‘HPMC’ refers to Hydroxypropyl Methylcellulose.

Term ‘HPC’ refers to Hydroxypropylcellulose.

Term ‘L-HPC’ refers to Low substitute Hydroxypropylcellulose

Term ‘MCC’ refers to Microcrystalline Cellulose.

Term ‘LOD’ refers to Loss on Drying.

Term ‘FBD’ refers to Fluidized Bed Dryer.

Term ‘IPA’ refers to Isopropyl alcohol.

Term ‘RPM’ refers to Revolutions per Minute.

Term ‘MDC’ refers to Methylene dichloride.

Term ‘HDPE’ refers to High Density Polyethylene.

Term ‘PVDC’ refers to Polyvinylidene dichloride.

Term ‘RPM’ refers to Rotation per minutes

### Immediate Release Composition:

In another embodiment, present invention relates to a pharmaceutical composition of Apremilast, Rifaximin is in the form of immediate release composition.

The objective of this invention is to provide a stable immediate release pharmaceutical composition comprising Apremilast and Rifaximin,

Immediate release pharmaceutical composition comprising Apremilast, Rifaximin, suitable binder, suitable extra granular agent and optionally with other necessary pharmaceutically acceptable ingredients/excipients.

The binder used can be selected from polyvinylpyrrolidone, hydroxypropylcellulose, hydroxypropylmethylcellulose, hydroxyethylcellulose, methylcellulose, sodium carboxymethyl cellulose, calcium carboxymethylcellulose, calcium carboxymethyl cellulose and/or mixtures thereof.

The ‘extra granular agent’ can be selected from cross-linked polymers: cross-linked polyvinylpyrrolidone (cross-povidone), cross-linked sodium carboxymethyl cellulose (croscarmellose sodium), L-HPC (hydroxypropylcellulose), Polacrillin Potassium and/or mixtures thereof.

The glidant used can be selected from silica gel or colloidal silicon dioxide, talc, magnesium carbonate and/or mixtures thereof.

The filler used is selected from dibasic calcium phosphate, lactose, dextrose, fructose, kaolin, sucrose, mannitol, microcrystalline cellulose, powdered cellulose, precipitated calcium carbonate, sorbitol, starch and/or mixtures thereof.

Suitable lubricant(s) can be selected from stearic acid, a metal salt of stearic acid such as magnesium stearate, talc, colloidal silica, a wax variety such as beads wax, spermaceti, boric acid, adipic acid, sodium sulphate, fumaric acid, stearyl sodium fumarate, sucrose aliphatic acid ester, a lauryl sulphate such as sodium lauryl sulphate, magnesium lauryl sulphate, starch derivative such as corn starch, potato starch, glyceryl behenate, behenoyl polyoxyl glyceride and/or mixtures thereof.

The film coating material used can be selected from polyvinylpyrrolidone, hydroxypropylcellulose, hydroxypropylmethylcellulose, hydroxyethylcellulose, methylcellulose, sodium carboxymethyl cellulose, calcium carboxymethylcellulose, calcium carboxymethyl cellulose and/or mixtures thereof.

Plasticizers can be selected from sorbitol, glycerol, triethylcitrate, polysorbate, carnauba wax, PEG (polyethylene glycol) and/or mixtures thereof.

In one of preferred embodiment, there is provided suitable range of each excipient in stable immediate release pharmaceutical composition as below:
a) Rifaximin in the range of 50.0 to 60.0% w/w;
b) Apremilast in the range of 1.0 to 2.5% w/w;
c) Binder in range of 1.5 to 5% w/w;
d) Glidant in the range of 1 to 2% w/w;
e) Lubricant in the range of 1 to 2% w/w;
f) Coating agent in the range of 5 to 10.0% w/w;
g) Filler in the range of 20 to 40% w/w

Preferred excipients for the stable immediate release pharmaceutical composition are as listed below:

The coating material is selected from Hydroxy Propyl Methyl Cellulose.

The binder is selected from Hydroxy Propyl Methyl Cellulose.

The glidant is selected from Colloidal Silicon Dioxides and purified talc.

The Lubricant is selected from various derivatives from stearates.

The plasticizer is PEG (Polyethylene glycol).

The filler is Microcrystalline Cellulose.

The stable pharmaceutical composition according to the present invention may be in the form of a tablet or a caplet or a capsule or a powder or a suspension in a liquid or an aerosol formulation or solutions, preferably in the form of a tablet or capsule.

### Delay Release Composition:

In another embodiment, present invention relates to a pharmaceutical composition of Apremilast, Rifaximin is in the form of delay release composition.

The objective of this invention is to provide a stable delay release pharmaceutical composition comprising Apremilast and Rifaximin,

Delay release pharmaceutical composition comprising Apremilast, Rifaximin, suitable disintegrant, suitable coating agent and optionally with other necessary pharmaceutically acceptable ingredients/excipients.

The ‘disintegrants’ can be selected from cross-linked polymers: cross-linked polyvinylpyrrolidone (cross-povidone), cross-linked sodium carboxymethyl cellulose (croscarmellose sodium), L-HPC (Low substitute hydroxypropylcellulose), Polacrillin Potassium and/or mixtures thereof.

The modified starch used is sodium starch glycolate.

The coating agent used is selected from methyl acrylate-methacrylic acid copolymers, cellulose acetate phthalate (CAP), Cellulose acetate succinate, hydroxypropyl methyl cellulose phthalate, hydroxypropyl methyl cellulose acetate succinate (hypromellose acetate succinate), polyvinyl acetate phthalate (PVAP), Methyl methacrylate-methacrylic acid copolymers and the like and suitable combination of thereof.

Delay release pharmaceutical composition comprising Apremilast, Rifaximin, suitable disintegrant, suitable filler, suitable binder, suitable glidant, suitable lubricant, suitable coating agent and suitable plasticizer.

The disintegrants and coating agent used is as define earlier.

The filler used is selected from dibasic calcium phosphate, lactose, dextrose, fructose, kaolin, sucrose, mannitol, microcrystalline cellulose, powdered cellulose, precipitated calcium carbonate, sorbitol, starch and/or mixtures thereof.

The binder used can be selected from polyvinylpyrrolidone, hydroxypropylcellulose, hydroxypropylnethylcellulose, hydroxyethylcellulose, methylcellulose, sodium carboxymethyl cellulose, calcium carboxymethylcellulose, calcium carboxymethyl cellulose and/or mixtures thereof.

The glidant used can be selected from silica gel or colloidal silicon dioxide, talc, magnesium carbonate and/or mixtures thereof.

Suitable lubricant(s) can be selected from stearic acid, a metal salt of stearic acid such as magnesium stearate, talc, colloidal silica, a wax variety such as beads wax, spermaceti, boric acid, adipic acid, sodium sulphate, fumaric acid, stearyl sodium fumarate, sucrose aliphatic acid ester, a lauryl sulphate such as sodium lauryl sulphate, magnesium lauryl sulphate, starch derivative such as corn starch, potato starch, glyceryl behenate, behenoyl polyoxyl glyceride and/or mixtures thereof.

Plasticizers can be selected from sorbitol, glycerol, triethylcitrate, polysorbate, carnauba wax, PEG (polyethylene glycol) and/or mixtures thereof.

In one of preferred embodiment, there is provided suitable range of each excipient in stable delay release pharmaceutical composition as below:
a) Rifaximin in the range of 50.0 to 60.0% w/w;
b) Apremilast in the range of 1.0 to 2.5% w/w;
c) Disintegrants in the range of 2.0 to 8% w/w;
d) Binder in range of 0.1 to 5% w/w;
e) Glidant in the range of 1 to 2% w/w;
g) Lubricant in the range of 1 to 2% w/w;
h) Coating agent 5 to 10.0% w/w;
i) Filler in the range of 20 to 40% w/w

Preferred excipients for the stable delay release pharmaceutical composition are as listed below:

The Disintegrants is selected from HPC (hydroxyl propyl cellulose) and Polacrillin Potassium.

The coating material is selected from methacrylic acid and its mixtures with other acrylates.

The filler is selected from microcrystalline cellulose, Lactose and suitable mixtures thereof.

The binder is selected from Povidone, HPMC and HPC and suitable mixtures thereof.

The glidant is selected from Colloidal Silicon Dioxides and purified talc.

The Lubricant is selected from various derivatives from stearates.

The plasticizer is PEG (Polyethylene glycol).

The stable pharmaceutical composition according to the present invention may be in the form of a tablet or a caplet or a capsule or a powder or a suspension in a liquid or an aerosol formulation or solutions, preferably in the form of a tablet or capsule.

In another embodiment, there is provided a process for the preparation of stable delay release pharmaceutical composition of Apremilast and Rifaximin comprises following steps:

General manufacturing Process for delay release tablet:
Apremilast and Rifaximin Tablets (10 mg + 500 mg)

| Ingredients/Inputs | Step/Process/Equipment |
|---|---|
| Co-sift Rifaximin, Apremilast, and MCC Sodium | 1. Sifting |
| through 40 mesh screen on sifter | (Sifter) |
| Add dispensed qty. of HPMC 6 cps under stirring until | 2. Binder solution |
| clear homogenous solution is formed | (Vessel with stirrer) |
| Load materials of step-1 in suitable capacity of RMG and | 3. Granulation |
| granulate the blend with step-2 binder solution | (RMG) |
| Dry the wet mass in FBD until desired LOD is achieved | 4. Drying |
| | (FBD) |
| Sift the dried granules through 20 mesh screen Mill the | 5. Sifting/Sizing |
| oversized granules through 1.0 mm screen fitted. | (Sifter/Co-mill) |
| Sift HPC and colloidal sillicon dioxide through 40 | 6. Sifting of Extra granular |
| mesh screen. | |
| Sift Magnesium Stearate through 60 mesh screen | |
| Blend the sized granules of step-5 with 40 mesh screen | 7. Blending |
| passed HPC and colloidal sillicon dioxide in suitable | (Blender) |
| capacity of blender. | |
| Lubricate the blend of step-7 in blender with 60 mesh | 8. Lubrication |
| screenm passed Magnesium Stearate. | (Blender) |
| Compress the blend of step-8 using D tooling puches and | 9. Compression |
| dies | (Compression Machine) |
| Take vessel and add dispensed qty. of IPA and | 10. Preparation of Film |
| Methacrylic acid copolymer Coating materials one by | Coating Solution |
| one under continuous stirring for 5 minutes. | (SS Container with stirrer) |
| Now add MDC under Continous Stirring for 45 min. | |
| Film Coating Parameters: | 11. Film Coating |
| Inlet Temp.: 40-75° C., Outlet Temp.: 35-80° C. | (Auto Coater) |
| Bed Temp.: 30-35° C., Pan RPM: 1-10 RPM | |
| Air Pressure: 1.0 3.0 Kg/cm². | |
| Spray Rate: 1 to 200 gm/min/gun, Peristaltic Pump | |
| Speed: 2-10 RPM | |
| | 12. Packing |

The resultant coated tablets are packed in PVDC. Alu Blisters or Alu Strip Packs.

### Gastro Resistance Followed by Controlled Release Composition:

In another embodiment, present invention relates to a pharmaceutical composition of Apremilast. Rifaximin is in the form of gastric resistance composition.

The term ‘Gastro resistance’ pharmaceutical composition means a pharmaceutical composition from which the drug may release from composition above certain pH to avoid gastric irritancy when taken orally. Such formulation may include enteric-coated tablets, enteric-coated pellets filled in capsules, enteric-coated mini tablets filled in capsule or enteric-coated capsules filled with tablets, pellets or powder. The term “Gastro resistance” or “Enteric Coated” is used herein to denote tablet of gastro resistance formulation which will not cause gastric irritancy when taken orally as compared to currently available immediate release capsule formulation in market. A single tablets can be taken with or without food over a period of 4 weeks. The amount will vary with the condition being treated; the stage of advancement of the condition, and the patient is adult or children.

The term ‘Gastro resistance’ or ‘Enteric Coated’ also means a composition in which the drug may be placed for drug Release above pH 5.5 to avoid gastric irritancy. Such formulation may include enteric coated tablets, enteric coated pellets filled in capsules, enteric coated mini tablets filled in capsule or enteric coated capsules filled with tablets, pellets or powder.

The objective of this invention is to provide a stable gastric resistance pharmaceutical composition comprising Apremilast, Rifaximin, suitable enteric coating material(s) and optionally with other necessary pharmaceutically ingredients/excipients.

The enteric coating material used is selected from methyl acrylate-methacrylic acid copolymers, Cellulose acetate phthalate (CAP), Cellulose acetate succinate, Hydroxypropyl methyl cellulose phthalate, Hydroxypropyl methyl cellulose acetate succinate (hypromellose acetate succinate), Polyvinyl acetate phthalate (PVAP), Methyl methacrylate-methacrylic acid copolymers and the like and suitable combination of thereof.

Optional pharmaceutically ingredients/excipients are selected from a disintegrant, a filler, a binder, a glidant, a lubricant and a plasticizer.

The most commonly used method of modulating the drug release is to include it in a matrix system. Hydrophilic polymer matrix systems are widely used in oral controlled drug delivery because of their flexibility to obtain a desirable drug release profile, cost-effectiveness, and broad regulatory acceptance. The drug release for extended duration, particularly for highly water-soluble drugs, using a hydrophilic matrix system is restricted due to rapid diffusion of the dissolved drug through the hydrophilic gel network.

Suitable hydrophilic polymers used according to the present invention include polyvinylpyrrolidone, hydroxypropyl cellulose, hydroxypropylmethyl cellulose, methylcellulose, vinyl acetate copolymers, polysaccharides such as alginate, xanthum gum and the like, polyethylene oxide, acrylic acid copolymers such as carbomer; maleic anhydride/methyl vinyl ether copolymers and derivatives and mixtures thereof.

The filler used is selected from dibasic calcium phosphate, lactose, dextrose, fructose, kaolin, sucrose, mannitol, microcrystalline cellulose, powdered cellulose, precipitated calcium carbonate, sorbitol, starch and/or mixtures thereof.

The binder used can be selected from polyvinylpyrrolidone, hydroxypropylcellulose, hydroxypropylnethylcellulose, hydroxyethylcellulose, methylcellulose, sodium carboxymethyl cellulose, calcium carboxymethylcellulose, calcium carboxymethyl cellulose and/or mixtures thereof.

The glidants used can be selected from silica gel or colloidal silicon dioxide, talc, magnesium carbonate and/or mixtures thereof.

Suitable lubricant(s) can be selected from stearic acid, a metal salt of stearic acid such as magnesium stearate, talc, colloidal silica, a wax variety such as beads wax, spermaceti, boric acid, adipic acid, sodium sulphate, fumaric acid, stearyl sodium fumarate, sucrose aliphatic acid ester, a lauryl sulphate such as sodium lauryl sulphate, magnesium lauryl sulphate, starch derivative such as corn starch, potato starch, glyceryl behenate, behenoyl polyoxyl glyceride and/or mixtures thereof.

Plasticizers can be selected from sorbitol, glycerol, triethylcitrate, polysorbate, carnauba wax, PEG (Polyethylene glycol) and/or mixtures thereof.

In one of the embodiment pharmaceutical composition of Apremilast and Rifaximin prepared by above process is stable.

In one of preferred embodiment, there is provided a range of each excipient in stable gastro resistant pharmaceutical composition as below:
a) Rifaximin in the range of 50.0 to 70.0% w/w;
b) Apremilast in the range of 1.0 to 2.5% w/w;
c) Binder in range of 0.1 to 5% w/w;
d) Glidant in the range of 1 to 2% w/w;
e) Lubricant in the range of 1 to 2% w/w;
g) Enteric Coating agent 5 to 10.0% w/w;
h) Filler in the range of 20 to 40% w/w

Preferred excipients for the stable gastro resistant pharmaceutical composition are as listed below:

The enteric coating material is selected from methacrylic acid and its mixtures with other acrylates.

The filler is selected from microcrystalline cellulose, Lactose and suitable mixtures thereof.

The binder is selected from Povidone, HPMC and HPC.

The glidant is selected from Colloidal Silicon Dioxides and purified talc.

The Lubricant is selected from various derivatives from stearates.

The plasticizer is PEG (Polyethylene glycol).

The stable pharmaceutical composition according to the present invention may be in the form of a tablet or a caplet or a capsule or a powder or a suspension in a liquid or an aerosol formulation or solutions, preferably in the form of a tablet or capsule.

In another embodiment, there is provided a process for the preparation of stable gastro resistant pharmaceutical composition of Apremilast and Rifaximin comprises following steps:

General Manufacturing Process for Gastric resitance tablet:
Apremilast and Rifaximin Tablets (10 mg + 500 mg)

| Ingredients/Inputs | Step/Process/Equipment |
|---|---|
| Co-sift Rifaximin, Apremilast, and MCC 101 and | 1. Sifting |
| HPMC through 40 mesh screen on vibratory sifter | (Sifter) |
| Dissolve HPC in dispensed qty. of Purified water | 2. Binder solution |
| under continuous stirring till clear solution obtained. | (SS Vessel with stirrer) |
| Load materials of step-1 in suitable capacity of RMG | 3. Granulation |
| and granulate the blend with step-2 binder solution | (RMG) |
| Dry the wet mass in FBD until desired LOD is achieved | 4. Drying |
| | (FBD) |
| Sift the dried granules through 20 mesh screen. Mill the | 5. Sifting/Sizing |
| oversized granules through 1.0 mm screen fitted. | (Sifter/Co-mill) |
| Sift colloidal sillicon dioxide through 40# sieve. | 6. Sifting of Extra granular |
| Sift Magnesium Stearate through 60# sieve. | |
| Blend the sized granules of step-5 with colloidal sillicon | 7. Blending |
| dioxide in suitable capacity of blender. | (Blender) |
| Lubricate the blend of step-7 in blender with 60# sieve | 8. Lubrication |
| passed Magnesium Stearate. | (Blender) |
| Compress the blend of step-8 using D tooling puches | 9. Compression |
| and dies | (Rotary Compression Machine) |
| Take vessel and add dispensed qty. of IPA and | 10. Preparation of Film Coating |
| disperse gastric resistance coating materials one by one | Solution |
| under continuous stirring for 5 minutes. | (SS Container with stirrer) |
| Now add MDC under Continous Stirring for 45 min. | |
| Film Coating Parameters: | 11. Film Coating |
| Inlet Temp.: 40-75° C., Outlet Temp.: 25-80° C. Bed | (Auto Coater) |
| Temp.: 30-55° C., Pan RPM: 1-10 RPM, Air Pressure: | |
| 1.0-3.0 Kg/cm². | |
| Spray Rate: 1 to 200 gm/min/gun, Peristaltic Pump | |
| Speed: 2-10 RPM | |
| | 12. Packing |
| | (PVDC Blister) |

The resultant coated tablets are packed in PVDC, Alu Blisters or Alu Strip Packs.

### Pharmaceutical Kit

‘Pharmaceutical Kit’ or “Combi pack” is intended to mean a kit made up of plastic or metallized paper/blister or other suitable ingredients known in the art which is used to keep a drug dosage safe from moisture, sunlight, microbial and dirt contamination.

‘Cavity’ means a casing or a place of medicament with specific size and shape in pharmaceutical kit.

‘Perforation’ means a specific pattern of holes done by piercing or any other method to separate/divide pharmaceutical kit in two or more parts.

The object of the invention is to provide a pharmaceutical kit comprising two parts divided with perforation or a punch or other suitable means having medicaments selected from Apremilast and Rifaximin. Wherein Apremilast and Rifaximin is provided in therapeutically effective amount of each of them. Therapeutically effective amount of Apremilast is selected from range of 1.00 mg to 100.00 mg. The preferred amount of Apremilast is selected from range of 10.00 mg to 70.00 mg. The most preferred amount of Apremilast is selected from range of 10.00 mg to 30.00 mg.

Therapeutically effective amount of Rifaximin is selected from range of 1.00 mg to 550.00 mg. The preferred amount of Rifaximin is selected from range of 250.00 mg to 500.00 mg. In more preferred embodiment the amount of Rifaximin is selected from range of 400.00 mg to 500.00 mg. The said therapeutically effective doses of Rifaximin and Apremilast meant to be used for human being in need thereof.

A specific object of the invention is to provide a pharmaceutical kit comprising two parts divided with perforation, wherein one part comprising a Aprimilast or its pharmaceutically acceptable salts and another part comprising Rifaximin.

In an object of the present invention is to provide a pharmaceutical kit is made up of primary packaging material.

Suitable primary packaging material may be selected from bottles, blisters, strips, sachet etc. For present invention strip or blister are suitable for pharmaceutical kit.

Materials for preparing strips or blisters are selected from aluminum foil, clear PVC/PVdC blister, amber/opaque PVC/PVdC blister. Materials are selected according to the physico-chemical characteristics of drug substances and drug product stability at different storage conditions.

Strips/Blisters are designed in such a way that cavities remain well separated/divided from each other. For separation purpose, strips/blisters are perforated or punched. Further, cavities of the blister are sealed with aluminum foil and this aluminum foil is printed with necessary information related to doses of medication.

Pharmaceutical kit of present invention wherein kit is divided by perforation wherein one part containing single cavity for one class of medicament and second part containing one or more than one cavities for other class of medicament.

In an object of the invention is to provide a pharmaceutical kit comprising medicaments are in tablet form.

In an object of the present invention, a pharmaceutical kit comprising medicament in suitable pharmaceutically acceptable formulation. Suitable pharmaceutically acceptable formulation of medicaments are selected from Delay release, immediate release and Gastric resistance.

In a specific object of present invention, tablets of Apremilast and Rifaximin or its pharmaceutically acceptable salts is in immediate release tablet form

The pharmaceutically acceptable excipients immediate release tablet is as define above.

In one of the specific object of the invention is to provide a pharmaceutical kit comprising two parts divided with perforation or punch or other suitable means, wherein one part comprising a Apremilast, either as a tablet of 15 mg or 25 mg and another part comprising Rifaximin as in tablet form of 500 mg used in combination therapy for treating IBD.

Present invention describes a pharmaceutical kit comprising two parts divided by perforation. Following are the examples by which active pharmaceutical ingredients can be formulated and further stored in pharmaceutical kit.

The pharmaceutical composition according to the present invention may be in the form of a tablet or capsule or a powder or a suspension in a liquid or an aerosol formulation or solutions.

In yet another embodiment, the combination or pharmaceutical composition of Apremilast and Rifaximin described as above is useful for the treatment of inflammatory bowel disease (IBD) wherein IBD if further classified to Ulcerative colitis (UC) and Crohn's disease.

In another embodiment, present invention relates to method of treating inflammatory bowel disease using a combination or pharmaceutical composition comprising Apremilast and Rifaximin.

In an embodiment the combination and pharmaceutical composition as mentioned above, is for use in combination with at least one suitable therapeutic agent.

Suitable therapeutic agent is selected from mesalamine, suitable corticosteroids, suitable immuno-suppressants and biologics therapy.

The invention is further exemplified by the following non-limiting examples, which are illustrative representing the preferred modes of carrying out the invention. The invention's scope is not limited to these specific embodiments only but should be read in conjunction with what is disclosed anywhere else in the specification together with those information and knowledge which are within the general understanding of a person skilled in the art.

The compositions are prepared and formulated according to conventional methods, such as those disclosed in standard reference texts and are well within the scope of a skilled person.

### Example 1: Gastric Resistance Composition of Apremilast and Rifaximin Tablets (15 mg+500 mg or 25 mg+500 mg)

TABLE 1
Composition of Gastric resistance
Apremilast and Rifaximin Tablets

| | | |
|---|---|---|
| Sr No. | Ingredients | Range % w/w |

Intragranular

| | | |
|---|---|---|
| 1 | Rifaximin | 50.0 to 70.0 |
| 2 | Apremilast | 1.0 to 2.5 |
| 3 | Microcrystalline Cellulose | 3.0 to 10.0 |
| 4 | Hydroxy Propyl Methyl Cellulose | 20.0 to 40.0 |

Binder Solution

| | | |
|---|---|---|
| 5 | Hydroxy Propyl Cellulose | 0.1 to 5 |
| 6 | Purified Water* | q.s. |

Extra granular

| | | |
|---|---|---|
| 7 | Colloidal Silicon dioxide | 1.0 to 2.0 |
| 8 | Magnesium Stearate | 1.0 to 2.0 |

Seal Coating

| | | |
|---|---|---|
| 9 | Hydroxy Propyl Methyl Cellulose | 5.0 to 20.0 |
| 10 | Polyethylene Glycol | 0.90 to 1.5 |
| 11 | Titanium Dioxide | 0.75 to 1.75 |
| 12 | Isopropyl Alcohol* | q.s. |
| 13 | Methylene Chloride* | q.s. |

Gastric resistance Coating

| | | |
|---|---|---|
| 14 | Methacrylic Acid-Methyl Acrylate | 5.0 to 20.0 |
| | Copolymer | |
| 15 | Polyethylene Glycol | 0.90 to 1.5 |
| 16 | Titanium Dioxide | 0.75 to 1.75 |
| 17 | Red Iron Oxide | 0.3 to 0.7 |
| 18 | Isopropyl Alcohol* | q.s. |
| 19 | Methylene Chloride* | q.s. |

*Does not remain in final product except in traces.

### Brief Manufacturing Process:

1. All intragranular materials were sifted from 20 #sieve.
2. Above sifted ingredients were mixed in RMG.
3. Dissolve Hydroxy Propyl Cellulose in purified water.
4. Dry mix mass of step 2 was granulated using solution of step 3 in RMG.
5. Wet mass was dried in dryer to obtain desired LOD.
6. Dried granules were sifted and milled to get granules of desired size.
7. Above sized granules of step 6 were mixed with sifted extra granular material and lubricated with sifted Magnesium stearate in a blender.
8. Compressed granular powder of step 7 in to tablets using appropriate tool.
9. Tablets of step 8, were coated with seal coating solution containing film former and other excipients.
10. Tablets of step 9, were coated with gastric resistant polymer coating solution containing polymer and other excipients.

### Example 2

Delay release composition of Apremilast and Rifaximin Tablets (15 mg+500 mg or 25 mg+500 mg)

TABLE 2
Composition of Delayed release Apremilast and Rifaximin Tablets

| | | |
|---|---|---|
| Sr No. | Ingredients | range % w/w |

Intragranular

| | | |
|---|---|---|
| 1 | Rifaximin | 50 to 60 |
| 2 | Apremilast | 1.0 to 2.5 |
| 3 | Microcrystalline Cellulose | 20 to 40 |

Binder Solution

| | | |
|---|---|---|
| 4 | Hydroxy Propyl Methyl Cellulose | 1.5 to 5.0 |
| 5 | Purified Water* | q.s. |

Extragranular

| | | |
|---|---|---|
| 6 | Hydroxypropyl Cellulose | 2.0 to 8.0 |
| 7 | Colloidal Silicon dioxide | 1 .0 to 2.0 |
| 8 | Magnesium Stearate | 1.0 to 2.0 |

Seal Coating

| | | |
|---|---|---|
| 9 | Hydroxy Propyl Methyl Cellulose | 5.0 to 20.0 |
| 10 | Polyethylene Glycol | 0.90 to 1.5 |
| 11 | Titanium Dioxide | 0.75 to 1.75 |
| 12 | Isopropyl Alcohol* | q.s. |
| 13 | Methylene Chloride* | q.s. |

Polymer Coating

| | | |
|---|---|---|
| 14 | Methacrylic acid copolymer | 5.0 to 20.0 |
| 15 | Polyethylene Glycol | 1.0 to 3.0 |
| 16 | Titanium Dioxide | 0.75 to 1.75 |
| 17 | Red Iron Oxide | 0.3 to 0.7 |
| 18 | Isopropyl Alcohol* | q.s. |
| 19 | Methylene Chloride* | q.s. |

*Does not remain in final product except in traces.

### Brief Manufacturing Process:

1. All intragranular materials were sifted from 20 #sieve.
2. Above sifted ingredients were mixed in RMG.
3. Dissolve Hydroxy Propyl Methyl Cellulose in purified water.
4. Dry mix mass of step 2 was granulated using solution of step 3 in RMG.
5. Wet mass was dried in dryer to obtain desired LOD.
6. Dried granules were sifted and milled to get granules of desired size.
7. Above sized granules of step 6 were mixed with sifted extragranular material and lubricated with sifted Magnesium stearate in a blender.
8. Compressed granular powder of step 7 in to tablets using appropriate tool.
9. Tablets of step 8, were coated with seal coating solution containing film former and other excipients.
10. Tablets of step 9, were coated with gastric resistant polymer coating solution containing polymer and other excipients.

### Example 3

Immediate release composition of Apremilast and Rifaximin Tablets (15 mg+500 mg or 25 mg+500 mg)

TABLE 3
Composition of Apremilast and Rifaximin Tablets

| Sr No. | Ingredients | Range % w/w |
|---|---|---|
| Intragranular | | |
| 1 | Rifaximin | 50 to 60 |
| 2 | Apremilast | 1.0 to 2.5 |
| 3 | Microcrystalline Cellulose | 20 to 40 |
| Binder Solution | | |
| 4 | Hydroxy Propyl Methyl Cellulose | 1.5 to 5 |
| 5 | Purified Water* | q.s. |
| Extra granular | | |
| 6 | Croscarmellose Sodium | 2.0 to 8.0 |
| 7 | Colloidal Silicon dioxide | 1 .0 to 2.0 |
| 8 | Magnesium Stearate | 1.0 to 2.0 |
| Film Coating | | |
| 9 | Hydroxy Propyl Methyl Cellulose | 5.0 to 10.0 |
| 10 | Polyethylene Glycol | 1.0 to 3.0 |
| 11 | Titanium Dioxide | 0.75 to 1.75 |
| 12 | Colorant | 0.3 to 0.7 |
| 13 | Isopropyl Alcohol* | q.s. |
| 14 | Methylene Chloride* | q.s. |

### Brief Manufacturing Process:

1. All intragranular materials were sifted from 20 #sieve.
2. Above sifted ingredients were mixed in RMG.
3. Dissolve Hydroxy Propyl Methyl Cellulose in purified water.
4. Dry mix mass of step 2 was granulated using solution of step 3 in RMG.
5. Wet mass was dried in dryer to obtain desired LOD.
6. Dried granules were sifted and milled to get granules of desired size.
7. Above sized granules of step 6 were mixed with sifted extragranular material and lubricated with sifted Magnesium stearate in blender.
8. Compressed granular powder of step 7 in to tablets using appropriate tool.
9. Tablets of step 8, were coated with seal coating solution containing film former and other excipients.

### Example 4

### Combi Pack of Apremilast Gastric Resistance Tablets and Rifaximin Gastric Resistance Tablets (15 mg & 500 mg and 25 mg & 500 mg)

TABLE 4A
Composition for Apremilast Gastric resistance Tablets

| | | |
|---|---|---|
| Sr No. | Ingredients | range % w/w |

Intragranular

| | | |
|---|---|---|
| 1 | Apremilast | 1.0 to 2.5 |
| 2 | Microcrystalline Cellulose | 3.0 to 10.0 |
| 3 | Hydroxy Propyl Methyl Cellulose | 20.0 to 40.0 |

Binder Solution

| | | |
|---|---|---|
| 4 | Hydroxy Propyl Cellulose | 0.1 to 5 |
| 5 | Purified Water* | q.s. |

Extra granular

| | | |
|---|---|---|
| 6 | Colloidal Silicon dioxide | 1 .0 to 2.0 |
| 7 | Magnesium Stearate | 1.0 to 2.0 |

Seal Coating

| | | |
|---|---|---|
| 8 | Hydroxy Propyl Methyl Cellulose | 5.0 to 20.0 |
| 9 | Polyethylene Glycol | 0.90 to 1.5 |
| 10 | Titanium Dioxide | 0.75 to 1.75 |
| 11 | Isopropyl Alcohol* | q.s. |
| 12 | Methylene Chloride* | q.s. |

Gastric resistance Coating

| | | |
|---|---|---|
| 13 | Methacrylic Acid-Methyl Acrylate | 5.0 to 20.0 |
| | Copolymer | |
| 14 | Polyethylene Glycol | 0.90 to 1.5 |
| 15 | Titanium Dioxide | 0.75 to 1.75 |
| 16 | Red Iron Oxide | 0.3 to 0.7 |
| 17 | Isopropyl Alcohol* | q.s. |
| 18 | Methylene Chloride* | q.s. |

*Does not remain in final product except in traces.

Brief manufacturing process (For Apremilast Gastric resistance tablets):
1. All intragranular materials were sifted from 20 #sieve.
2. Above sifted ingredients were mixed in RMG.
3. Dissolve Hydroxy Propyl Cellulose in purified water.
4. Dry mix mass of step 2 was granulated using solution of step 3 in RMG.
5. Wet mass was dried in dryer to obtain desired LOD.
6. Dried granules were sifted and milled to get granules of desired size.
7. Above sized granules of step 6 were mixed with sifted extragranular material and lubricated with sifted Magnesium stearate in a blender.
8. Compressed granular powder of step 7 in to tablets using appropriate tool.
9. Tablets of step 8, were coated with seal coating solution containing film former and other excipients.
10. Tablets of step 9, were coated with gastric resistant polymer coating solution containing polymer and other excipients.

TABLE 4B
Composition for Rifaximin Gastric resistance Tablets:

| | | |
|---|---|---|
| Sr No. | Ingredients | range % w/w |

Intragranular

| | | |
|---|---|---|
| 1 | Rifaximin | 50.0 to 70.0 |
| 2 | Microcrystalline Cellulose | 3.0 to 10.0 |
| 3 | Hydroxy Propyl Methyl Cellulose | 20.0 to 40.0 |

Binder Solution

| | | |
|---|---|---|
| 4 | Hydroxy Propyl Cellulose | 0.1 to 5 |
| 5 | Purified Water* | q.s. |

Extra granular

| | | |
|---|---|---|
| 6 | Colloidal Silicon dioxide | 1.0 to 2.0 |
| 7 | Magnesium Stearate | 1.0 to 2.0 |

Seal Coating

| | | |
|---|---|---|
| 8 | Hydroxy Propyl Methyl Cellulose | 5.0 to 20.0 |
| 9 | Polyethylene Glycol | 0.90 to 1.5 |
| 10 | Titanium Dioxide | 0.75 to 1.75 |
| 11 | Isopropyl Alcohol* | q.s. |
| 12 | Methylene Chloride* | q.s. |

Gastric resistance Coating

| | | |
|---|---|---|
| 13 | Methacrylic Acid-Methyl Acrylate | 5.0 to 20.0 |
| | Copolymer | |
| 14 | Polyethylene Glycol | 0.90 to 1.5 |
| 15 | Titanium Dioxide | 0.75 to 1.75 |
| 16 | Red Iron Oxide | 0.3 to 0.7 |
| 17 | Isopropyl Alcohol* | q.s. |
| 18 | Methylene Chloride* | q.s. |

*Does not remain in final product except in traces.

### Brief Manufacturing Process (for Rifaximin Gastric Resistance Tablets):

1. All intragranular materials were sifted from 20 #sieve.
2. Above sifted ingredients were mixed in RMG.
3. Dissolve Hydroxy Propyl Cellulose in purified water.
4. Dry mix mass of step 2 was granulated using solution of step 3 in RMG.
5. Wet mass was dried in dryer to obtain desired LOD.
6. Dried granules were sifted and milled to get granules of desired size.
7. Above sized granules of step 6 were mixed with sifted extragranular material and lubricated with sifted Magnesium stearate in a blender.
8. Compressed granular powder of step 7 in to tablets using appropriate tool.
9. Tablets of step 8, were coated with seal coating solution containing film former and other excipients.
10. Tablets of step 9, were coated with gastric resistant polymer coating solution containing polymer and other excipients.
11. Apremilast GR tablet/s and Rifaximin GR tablet/s are co-pack in Alu-alublister.

### Example 5

### Combi Pack of Apremilast Delay Release Tablets and Rifaximin Delay Release Tablets (15 mg & 500 mg and 25 mg & 500 mg)

TABLE 5A
Composition of Apremilast Delayed release tablets

| | | |
|---|---|---|
| Sr No. | Ingredients | range % w/w |

Intragranular

| | | |
|---|---|---|
| 1 | Apremilast | 1.0 to 2.5 |
| 2 | Microcrystalline Cellulose | 20 to 40 |

Binder Solution

| | | |
|---|---|---|
| 3 | Hydroxy Propyl Methyl Cellulose | 1.5 to 5 |
| 4 | Purified Water* | q.s. |

Extra granular

| | | |
|---|---|---|
| 5 | Hydroxypropyl Cellulose | 2.0 to 8.0 |
| 6 | Colloidal Silicon dioxide | 1 .0 to 2.0 |
| 7 | Magnesium Stearate | 1.0 to 2.0 |

Seal Coating

| | | |
|---|---|---|
| 8 | Hydroxy Propyl Methyl Cellulose | 5.0 to 20.0 |
| 9 | Polyethylene Glycol | 0.90 to 1.5 |
| 10 | Titanium Dioxide | 0.75 to 1.75 |
| 11 | Isopropyl Alcohol* | q.s. |
| 12 | Methylene Chloride* | q.s. |

Polymer Coating

| | | |
|---|---|---|
| 13 | Methacrylic acid copolymer | 5.0 to 20.0 |
| 14 | Polyethylene Glycol | 1.0 to 3.0 |
| 15 | Titanium Dioxide | 0.75 to 1.75 |
| 16 | Red Iron Oxide | 0.3 to 0.7 |
| 17 | Isopropyl Alcohol* | q.s. |
| 18 | Methylene Chloride* | q.s. |

*Does not remain in final product except in traces.

### Brief Manufacturing Process: (for Apremilast Delay Release Tablets)

1. All intragranular materials were sifted from 20 #sieve.
2. Above sifted ingredients were mixed in RMG.
3. Dissolve Hydroxy Propyl Methyl Cellulose in purified water.
4. Dry mix mass of step 2 was granulated using solution of step 3 in RMG.
5. Wet mass was dried in dryer to obtain desired LOD.
6. Dried granules were sifted and milled to get granules of desired size.
7. Above sized granules of step 6 were mixed with sifted Extragranular material and lubricated with sifted Magnesium stearate in a blender.
8. Compressed granular powder of step 7 in to tablets using appropriate tool.
9. Tablets of step 8, were coated with seal coating solution containing film former and other excipients.
10. Tablets of step 9, were coated with delayed release polymer coating solution containing polymer and other excipients.

TABLE 5B
Composition of Rifaximin Delayed release tablets

| | | |
|---|---|---|
| Sr No. | Ingredients | Range % w/w |

Intragranular

| | | |
|---|---|---|
| 1 | Rifaximin | 50 to 60 |
| 2 | Microcrystalline Cellulose | 20 to 40 |

Binder Solution

| | | |
|---|---|---|
| 3 | Hydroxy Propyl Methyl Cellulose | 1.5 to 5 |
| 4 | Purified Water* | q.s. |

Extra granular

| | | |
|---|---|---|
| 5 | Hydroxypropyl Cellulose | 2.0 to 8.0 |
| 6 | Colloidal Silicon dioxide | 1.0 to 2.0 |
| 7 | Magnesium Stearate | 1.0 to 2.0 |

Seal Coating

| | | |
|---|---|---|
| 8 | Hydroxy Propyl Methyl Cellulose | 5.0 to 20.0 |
| 9 | Polyethylene Glycol | 0.90 to 1.5 |
| 10 | Titanium Dioxide | 0.75 to 1.75 |
| 11 | Isopropyl Alcohol* | q.s. |
| 12 | Methylene Chloride* | q.s. |

Polymer Coating

| | | |
|---|---|---|
| 13 | Methacrylic acid copolymer | 5.0 to 20.0 |
| 14 | Polyethylene Glycol | 1.0 to 3.0 |
| 15 | Titanium Dioxide | 0.75 to 1.75 |
| 16 | Red Iron Oxide | 0.3 to 0.7 |
| 17 | Isopropyl Alcohol* | q.s. |
| 18 | Methylene Chloride* | q.s. |

*Does not remain in final product except in traces.

### Brief Manufacturing Process: (for Rifaximin Delay Release Tablets)

1. All intragranular materials were sifted from 20 #sieve.
2. Above sifted ingredients were mixed in RMG.
3. Dissolve Hydroxy Propyl Methyl Cellulose in purified water.
4. Dry mix mass of step 2 was granulated using solution of step 3 in RMG.
5. Wet mass was dried in dryer to obtain desired LOD.
6. Dried granules were sifted and milled to get granules of desired size.
7. Above sized granules of step 6 were mixed with sifted extragranular material and lubricated with sifted Magnesium stearate in a blender.
8. Compressed granular powder of step 7 in to tablets using appropriate tool.
9. Tablets of step 8, were coated with seal coating solution containing film former and other excipients.
10. Tablets of step 9, were coated with delayed release polymer coating solution containing polymer and other excipients.
11. Apremilast delayed release tablet/s and Rifaximin delayed release tablet/s are co-packed in Alu-alu blister/PVDC blister.

### Example 6

### Combi Pack of Apremilast Immediate Release Tablets and Rifaximin Immediate Release Tablets (15 mg & 500 mg and 25 mg & 500 mg)

TABLE 6A
Composition of Apremilast Tablets

| | | |
|---|---|---|
| Sr No. | Ingredients | Range % w/w |

Intragranular

| | | |
|---|---|---|
| 1 | Apremilast | 1.0 to 2.5 |
| 2 | Microcrystalline Cellulose | 20 to 40 |

Binder Solution

| | | |
|---|---|---|
| 3 | Hydroxy Propyl Methyl Cellulose | 1.5 to 5 |
| 4 | Purified Water* | q.s. |

Extra granular

| | | |
|---|---|---|
| 5 | Crosscarmellose Sodium | 2.0 to 8.0 |
| 6 | Colloidal Silicon dioxide | 1 .0 to 2.0 |
| 7 | Magnesium Stearate | 1.0 to 2.0 |

Film Coating

| | | |
|---|---|---|
| 8 | Hydroxy Propyl Methyl Cellulose | 5.0 to 10.0 |
| 9 | Polyethylene Glycol | 1.0 to 3.0 |
| 10 | Titanium Dioxide | 0.75 to 1.75 |
| 11 | Colorant | 0.3 to 0.7 |
| 12 | Isopropyl Alcohol* | q.s. |
| 13 | Methylene Chloride* | q.s. |

*Does not remain in final product except in traces.

### Brief Manufacturing Process: (for Apremilast Immediate Release Tablets)

1. All intragranular materials were sifted from 20 #sieve.
2. Above sifted ingredients were mixed in RMG.
3. Dissolve Hydroxy Propyl Methyl Cellulose in purified water.
4. Dry mix mass of step 2 was granulated using solution of step 3 in RMG.
5. Wet mass was dried in dryer to obtain desired LOD.
6. Dried granules were sifted and milled to get granules of desired size.
7. Above sized granules of step 6 were mixed with sifted Extragranular material and lubricated with sifted Magnesium stearate in blender.
8. Compressed granular powder of step 7 in to tablets using appropriate tool.
9. Tablets of step 8, were coated with seal coating solution containing film former and other excipients.

TABLE 6B
Composition of Rifaximin tablets

| | | |
|---|---|---|
| Sr No. | Ingredients | Range % w/w |

Intragranular

| | | |
|---|---|---|
| 1 | Rifaximin | 50 to 60 |
| 2 | Microcrystalline Cellulose | 20 to 40 |

Binder Solution

| | | |
|---|---|---|
| 3 | Hydroxy Propyl Methyl Cellulose | 1.5 to 5 |
| 4 | Purified Water* | q.s. |

Extra granular

| | | |
|---|---|---|
| 5 | Crosscarmellose Sodium | 2.0 to 8.0 |
| 6 | Colloidal Silicon dioxide | 1 .0 to 2.0 |
| 7 | Magnesium Stearate | 1.0 to 2.0 |

Film Coating

| | | |
|---|---|---|
| 8 | Hydroxy Propyl Methyl Cellulose | 5.0 to 10.0 |
| 9 | Polyethylene Glycol | 1.0 to 3.0 |
| 10 | Titanium Dioxide | 0.75 to 1.75 |
| 11 | Colorant | 0.3 to 0.7 |
| 12 | Isopropyl Alcohol* | q.s. |
| 13 | Methylene Chloride* | q.s. |

*Does not remain in final product except in traces

### Brief Manufacturing Process: (for Rifaximin Immediate Release Tablets)

1. All intergranular materials were sifted from 20 #sieve.
2. Above sifted ingredients were mixed in RMG.
3. Dissolve HPMC 6 cps in purified water.
4. Dry mix mass of step 2 was granulated using solution of step 3 in RMG.
5. Wet mass was dried in fluid bed dryer to obtain desired LOD.
6. Dried granules were sifted and milled to get granules of desired size.
7. Above sized granules of step 6 were mixed with sifted extragranular material and lubricated with sifted Magnesium stearate in blender.
8. Compressed granular powder of step 7 in to tablets using appropriate tool.
9. Tablets of step 8, were coated with seal coating solution containing film former and other excipients.
10. Apremilast tablet/s and Rifaximin tablet/s are co-packed in Alu-alu blister/PVDC blister.

### Pharmacological Studies

### Example 7

### Evaluation of Apremilast and Rifaximin Alone and in Combination in DSS Induced Ulcerative Colitis in C57 Mice Model.

In this study, we have used human equivalent mice dose of Apremilast and very ( 1/18^{th}-fold) low dose of Rifaximin in animal model, which is DSS (Dextran sulfate sodium) induced ulcerative colitis in C57 mice (Br J Pharmacol. 2019 July; 176 (13): 2209-2226. doi: 10.1111/bph.14667. Epub 2019 May 17). The colitis induced by DSS, presents good reproducibility and also mimics clinical symptoms, inflammatory markers and histopathological features which is similar to IBD in humans (Cell Mol Gastroenterol Hepatol. 2015 Mar. 1; 1 (2): 154-170. doi: 10.1016/j.jcmgh.2015.01.006., Arq Gastroenterol. 2014 April-June; 51 (2): 107-12. doi: 10.1590/s0004-28032014000200007. PMID: 25003261).

### Methods

C57 mice of 10-12 weeks age were used for this study. On the day of study initiation (day-0) animals were randomized based on their body weight in to different treatment groups as below:

TABLE 7
Treatment groups and dose levels

| Sr. | | | No. of |
|---|---|---|---|
| no. | Treatment group | Dose and route | animals |
| 1 | Water Vehicle Control | 0 mg/kg, p.o. | 06 |
| 2 | DSS (2.5%) vehicle control | 0 mg/kg, p.o. | 10 |
| 3 | Rifaximin | 19 mg/kg, p.o. | 10 |
| 4 | Apremilast | 12.5 mg/kg, p.o. | 10 |
| 5 | Rifaximin (19 mg/kg, | Rifaximin-19 mg/kg, | 10 |
| | p.o.) + Apremilast | p.o. + Apremilast- | |
| | (12.5 mg/kg. p.o.) | 12.5 mg/kg, p.o. | |

On day-0 animals were randomized based on body weight and from the same day onwards animals were administered with 2.5% DSS in RO water along with the test compounds till day-6.

The test compounds were formulated in Tween 80 and 0.5% Na. CMC in (0.5:99.50%) ratio and oral administration was done once a day for six days. During the duration of the experiment, a disease activity index (DAI) score was assessed to evaluate the clinical progression of ulcerative colitis. The DAI was calculated by grading on a scale of 0-5 using the following parameters: loss of body weight (0=normal; 1=0-5%; 2=6-10%; 3=11-15%; 4=16-20%, 5=>20%), stool consistency (0=normal; 1=soft stools; 2=loose stools; 3=watery diarrhea) and rectal bleeding (0=none; 2=presence of hemoccult; 4=severe bleeding). (Ref: Cytokine 2014, 66, 30-39).

On the day of termination (Day-6), DAI score was assessed followed by nonfasted blood collection. Then animals were sacrificed and relevant tissues were collected in 10% formalin for histological analysis or snap frozen in liquid nitrogen for other assays.

With the help of MS Excel, percent change vs disease control (DSS vehicle control) in various parameters were calculated and statistical analysis was performed using graph pad prism software.

### Results

a) The disease activity index (DAI) score was assessed on day-6 to evaluate the clinical progression of ulcerative colitis as given in Table 8.

TABLE 8
The disease activity index (DAI) score

| | | | % change in |
|---|---|---|---|
| | | | DAI vs DSS |
| | | | vehicle |
| Sr. No. | Treatment groups | Day-6 | control |
| 1 | Water vehicle control | 0.7 ± 0.4 | |
| 2 | DSS (2.5%) vehicle | 6.1 ± 0.8 | |
| | control | | |
| 3 | Rifaximin | 5.9 ± 0.7 | −3.3 ± 11.8 |
| | (19 mg/kg, p.o.) | | |
| 4 | Apremilast | 4.1 ± 1.0 | −32.8 ± 16.7 |
| | (12.5 mg/kg, p.o.) | | |
| 5 | Rifaximin (19 mg/kg, | 2.1 ± 0.5** | −65.4 ± 7.9 |
| | p.o.) + Apremilast | | |
| | (12.5 mg/kg, p.o.) | | |

**Significantly different from DSS Vehicle control at p < 0.01 by using one-way ANOVA followed by Dunnett t-test. [n = 6 in Water vehicle control, n = 10 in DSS vehicle control, n = 10 Rifaximin in (19 mg/kg, p.o.), n = 10 Apremilast in (12.5 mg/kg, p.o.) and n = 10 in Rifaximin (19 mg/kg, p.o.) + Apremilast (12.5 mg/kg, p.o.)].

b) The rectal bleeding is one the most important feature of ulcerative colitis the scores in different treatment groups is as given in table 9.

TABLE 9
The rectal bleeding score

| | |
|---|---|
| | % change in |
| | bleeding |
| Sr. | score vs DSS |

| No. | Treatment groups | Day-6 | vehicle control |
|---|---|---|---|
| 1 | Water vehicle control | 0.00 ± 0.00 | |
| 2 | DSS (2.5%) | 2.8 ± 0.6 | |
| | vehicle control | | |

| | | | |
|---|---|---|---|
| 3 | Rifaximin | 1.7 ± 0.5 | −39.3 ± 16.9 |
| | (19 mg/kg, p.o.) | | |
| 4 | Apremilast | 1.2 ± 0.6 | −57.1 ± 21.8 |
| | (12.5 mg/kg, p.o.) | | |
| 5 | Rifaximin | 0.00 ± 0.00*** | −100.0 ± 0.0 |
| | (19 mg/kg, p.o.) + | | |
| | Apremilast | | |
| | (12.5 mg/kg, p.o.) | | |

***Significantly different from DSS Vehicle control at p < 0.001 by using one-way ANOVA followed by Dunnett t-test. [n = 6 in Water vehicle control, n = 10 in DSS vehicle control, n = 10 in Rifaximin (19 mg/kg, p.o.), n = 10 in Apremilast (12.5 mg/kg, p.o.) and n = 10 in Rifaximin (19 mg/kg, p.o.) + Apremilast (12.5 mg/kg, p.o.)].

### Observation:

Apremilast treatment showed 32.8% improvement in disease activity index whereas low dose of Rifaximin didn't show any improvement but when animals are treated with combination of Apremilast and Rifaximin, it showed statistically very significant 65.4% improvement in disease activity index (DAI) score. The DAI score comprises of grading for loss of body, stool consistency and rectal bleeding which is the prominent clinical disease parameters of evaluation in inflammatory bowel disease. When we evaluate the effect on rectal bleeding combination of Apremilast and Rifaximin has shown statistically very significant 100% improvement in rectal bleeding.

### Conclusion

Oral administration of Apremilast at human equivalent mice dose and very ( 1/18^{th}-fold) low dose of Rifaximin showed synergistic efficacy of combination as compared to alone Aprimilast or Rifaximin in DSS induced ulcerative colitis in C57 mice, a model of inflammatory bowel disease.

### Example 8

### Evaluation of Apremilast and Rifaximin Alone and as Fixed Dose Combination (FDC) in DSS Induced Ulcerative Colitis in C57 Mice.

In this study, we have used Apremilast which is equivalent to 10 mg of human dose and Rifaximin which is equivalent to 500 mg of human dose in animal model, which is DSS (Dextran sulfate sodium) induced ulcerative colitis in C57 mice (Br J Pharmacol. 2019 July; 176 (13): 2209-2226. doi: 10.1111/bph.14667. Epub 2019 May 17). The colitis induced by

DSS, presents good reproducibility and also mimics clinical symptoms, inflammatory markers and histopathological features which is similar to IBD in humans (Cell Mol Gastroenterol Hepatol. 2015 Mar. 1; 1 (2): 154-170. doi: 10.1016/j.jcmgh.2015.01.006., Arq Gastroenterol. 2014 April-June; 51 (2): 107-12. doi: 10.1590/s0004-28032014000200007. PMID: 25003261).

### Methods

C57 mice of 9-10 weeks age were used for this study. On the day of study initiation (day-0) animals were randomized based on their body weight in to different treatment groups as below:

TABLE 10
Treatment groups and dose levels

| Sr. | | | No. of |
|---|---|---|---|
| no. | Treatment group | Dose and route | animals |
| 1 | Water Vehicle Control | 0 mg/kg, p.o. | 06 |
| 2 | DSS (2.5%) vehicle control | 0 mg/kg, p.o. | 10 |
| 3 | Rifaximin (102.8 mg/kg, p.o., | 102.8 mg/kg, p.o., BID | 10 |
| | BID) | (Equivalent human dose = 500 mg) | |
| 4 | Apremilast (2.1 mg/kg, p.o., | 2.1 mg/kg, p.o., BID | 10 |
| | BID) | (Equivalent human dose = 10 mg) | |
| 5 | Rifaximin (102.8 mg/kg, p.o., | 102.8 mg/kg, p.o., BID + | 10 |
| | BID) + Apremilast (2.1 mg/kg. | 2.1 mg/kg, p.o., BID | |
| | p.o., BID) | [Equivalent human dose of | |
| | | Rifaximin is 500 mg and | |
| | | Apremilast is 10 mg, BID] | |

On day-0 animals were randomized based on body weight and from the same day onwards animals were administered with 2.5% DSS in RO water along with the test compounds till day-6. The test compounds were formulated in Tween 80 and 0.5% Na. CMC in (0.5:99.50%) ratio and oral administration was done twice a day for six days. During the duration of the experiment, a disease activity index (DAI) score was assessed to evaluate the clinical progression of ulcerative colitis.

The DAI was calculated by grading on a scale of 0-5 using the following parameters: loss of body weight (0=normal; 1=0-5%; 2=6-10%; 3=11-15%; 4=16-20%, 5=>20%), stool consistency (0=normal; 1=soft stools; 2=loose stools; 3=watery diarrhea) and rectal bleeding (0=none; 2=presence of hemoccult; 4=severe bleeding). (Ref: Cytokine 2014, 66, 30-39).

On the day of termination (Day-6), DAI score was assessed followed by nonfasted blood collection. Then animals were sacrificed and relevant tissues were collected in 10% formalin for histological analysis or snap frozen in liquid nitrogen for other assays.

With the help of MS Excel, percent change vs disease control (DSS vehicle control) in various parameters were calculated and statistical analysis was performed using graph pad prism software.

### Results

a) The disease activity index (DAI) score was assessed on day-6 to evaluate the clinical progression of ulcerative colitis as given in Table 11.

TABLE 11
The disease activity index (DAI) score

| | |
|---|---|
| Sr. | % change in DAI vs |

| No. | Treatment groups | Day-6 | DSS vehicle control |
|---|---|---|---|
| 1 | Water Vehicle Control | 0.3 ± 0.2 | |
| 2 | DSS (2.5%) vehicle control | 7.8 ± 0.8 | |

| | | | |
|---|---|---|---|
| 3 | Rifaximin | 6.4 ± 0.9 | −17.9 ± 11.2 |
| | (102.8 mg/kg, p.o., BID) | | |
| 4 | Apremilast | 6.9 ± 0.7 | −11.5 ± 9.5 |
| | (2.1 mg/kg. p.o., BID) | | |
| 5 | Rifaximin (102.8 mg/kg, | 4.3 ± 0.6** | −44.4 ± 7.7 |
| | p.o., BID) + Apremilast | | |
| | (2.1 mg/kg. p.o., BID) | | |

**Significantly different from DSS Vehicle control at p < 0.01 by using one-way ANOVA followed by Dunnett t-test. [n = 6 in Water vehicle control, n = 10 in DSS vehicle control, n = 10 in Rifaximin (102.8 mg/kg, p.o., BID), n = 10 in Apremilast (2.1 mg/kg, p.o., BID) and n = 9 in Rifaximin (102.8 mg/kg, p.o., BID) + Apremilast (2.1 mg/kg, p.o., BID)].

b) The rectal bleeding is one the most important feature of ulcerative colitis the scores in different treatment groups is as given in table 12.

TABLE 12
The rectal bleeding score

% change in
bleeding

| Sr. | | | score vs DSS |
|---|---|---|---|
| No. | Treatment groups | Day-6 | vehicle control |
| 1 | Water Vehicle Control | 0.0 ± 0.0 | |
| 2 | DSS (2.5%) | 2.4 ± 0.4 | |
| | vehicle control | | |

| | | | |
|---|---|---|---|
| 3 | Rifaximin | 2.0 ± 0.5 | −16.7 ± 21.5 |
| | (102.8 mg/kg, p.o., BID) | | |
| 4 | Apremilast | 2.0 ± 0.4 | −16.7 ± 17.6 |
| | (2.1 mg/kg. p.o., BID) | | |
| 5 | Rifaximin | 0.2 ± 0.2*** | −90.7 ± 9.3 |
| | (102.8 mg/kg, p.o., | | |
| | BID) + Apremilast | | |
| | (2.1 mg/kg. p.o., BID) | | |

***Significantly different from DSS Vehicle control at p < 0.001 by using one-way ANOVA followed by Dunnett t-test. [n = 6 in Water vehicle control, n = 10 in DSS vehicle control, n = 10 in Rifaximin (102.8 mg/kg, p.o., BID), n = 10 in Apremilast (2.1 mg/kg, p.o., BID) and n = 9 in Rifaximin (102.8 mg/kg, p.o., BID) + Apremilast (2.1 mg/kg, p.o., BID)].

### Observation:

Apremilast showed 12% improvement and Rifaximin showed 18% improvement in disease activity index (DAI) score but when animals are treated with combination of Apremilast and Rifaximin, it showed statistically very significant 44% improvement in DAI score. When we evaluate the effect on rectal bleeding, combination of apremilast and rifaximin has shown statistically very significant 91% improvement in rectal bleeding which was 17% alone in Apremilast and Rifaximin treatment.

### Conclusion

Oral administration of Apremilast (equivalent to 10 mg human dose) and Rifaximin (equivalent to 500 mg human dose) showed synergistic efficacy of combination as compared to alone Aprimilast or Rifaximin in DSS induced ulcerative colitis in C57 mice, a model of inflammatory bowel disease.

### Example 9

### Evaluation of Apremilast and Rifaximin Combination in DSS Induced Ulcerative Colitis in Mdr2KO Mice, PSC-IBD Model.

Multidrug resistance protein 2 knockout (Mdr2KO: Mdr2−/−) mice develop spontaneous cholestatic liver injury and fibrosis mirroring human primary sclerosing cholangitis (PSC). Among PSC patients, approximately 75% will develop coexisting inflammatory bowel disease (IBD), predominantly in the form of ulcerative colitis (UC). In Mdr2KO model cholestasis-induced alterations in bile acid homeostasis are associated with hepatobiliary inflammation and progressive liver injury and as seen in human PSC. Alterations in bile acid homeostasis are likely to play a more relevant role in modulating disease severity in the setting of combined cholestasis and colitis as compared to colitis alone. Mdr2KO mouse in combination with chemically induced DSS colitis present a novel model of combined cholestatic liver injury and colitis demonstrating similar features to human PSC-IBD. (Mucosal Immunol. 2021 March; 14 (2): 479-490. doi: 10.1038/s41385-020-00347-6.). Patients with concurrent PSC and IBD have an increased risk of colorectal cancer (Gastroenterology 1996; 110:331; Hepatology 1995; 22:1404; Am J Gastroenterol 1999; 94:1643). In this study, we have used Apremilast which is equivalent to 10 and 30 mg and Rifaximin which is equivalent to 500 mg of human dose in animal model, which is DSS (Dextran sulfate sodium) induced ulcerative colitis in Mdr2KO mice which is model for PSC induced inflammatory bowel disease.

### Methods

Mdr2KO mice of 15-16 weeks age were used for this study. On the day of study initiation (day-0) animals were randomized based on their body weight in to different treatment groups as below:

TABLE 13
Treatment groups and dose levels

| Sr. | | | No. of |
|---|---|---|---|
| no. | Treatment group | Dose and route | animals |
| 1 | Water Vehicle Control | 0 mg/kg, p.o. | 06 |
| 2 | DSS (2.5%) vehicle control | 0 mg/kg, p.o. | 08 |
| 3 | Rifaximin (102.8 mg/kg, p.o., | 102.8 mg/kg, p.o., BID | 08 |
| | BID) + Apremilast (2.1 mg/kg, | (Equivalent human dose = 500 mg) + | |
| | p.o., BID) | 2.1 mg/kg, p.o., BID | |
| | | (Equivalent human dose = 10 mg) | |
| 4 | Rifaximin (102.8 mg/kg, p.o., | 102.8 mg/kg, p.o., BID | 08 |
| | BID) + Apremilast (6.2 mg/kg, | (Equivalent human dose = 500 mg) + | |
| | p.o., BID) | 6.2 mg/kg, p.o., BID | |
| | | (Equivalent human dose = 30 mg) | |

On day-0 animals were randomized based on body weight and from the same day onwards animals were administered with 2.5% DSS in RO water along with the test compounds till day-6.

The test compounds were formulated in Tween 80 and 0.5% Na. CMC in (0.5:99.50%) ratio and oral administration was done twice a day for six days. During the duration of the experiment, a disease activity index (DAI) score was assessed to evaluate the clinical progression of ulcerative colitis. The DAI was calculated by grading on a scale of 0-5 using the following parameters: loss of body weight (0=normal; 1=0-5%; 2=6-10%; 3=11-15%; 4=16-20%, 5=>20%), stool consistency (0=normal; 1=soft stools; 2=loose stools; 3=watery diarrhea) and rectal bleeding (0=none; 2=presence of hemoccult; 4=severe bleeding). (Ref: Cytokine 2014, 66, 30-39).

On the day of termination (Day-6), DAI score was assessed followed by nonfasted blood collection. Then animals were sacrificed, colon length was measured and then collected in 10% formalin for histological analysis or snap frozen in liquid nitrogen for other assays. For histological grades Hematoxylin and eosin (H&E) staining was performed for the confirmation of inflammation in the colon and Masson's trichrome staining was performed for the confirmation of collagen score (presence of fibrosis) in the colon tissue.

With the help of MS Excel, percent change vs disease control (DSS vehicle control) in various parameters were calculated and statistical analysis was performed using graph pad prism software. Percentage improvement of colon length was calculated by calculating the decrease in colon length due to disease from water vehicle control group and then improvement in colon length against DSS vehicle control group by considering the colon length of water vehicle control group as 100%.

### Results

a) The disease activity index (DAI) score was assessed on day-6 to evaluate the clinical progression of ulcerative colitis as given in Table 14.

TABLE 14
The disease activity index (DAI) score

% change

| | | | in DAI |
|---|---|---|---|
| | | | vs DSS |
| | | | vehicle |
| Sr. No. | Treatment groups | Day-6 | control |
| 1 | Water Vehicle Control | 0.7 ± 0.3 | |
| 2 | DSS (2.5%) | 11.0 ± 0.7 | |
| | vehicle control | | |

| | | | |
|---|---|---|---|
| 3 | Rifaximin (102.8 mg/kg, | 7.0 ± 0.7*** | −36.4 ± 6.4 |
| | p.o., BID) + Apremilast | | |
| | (2.1 mg/kg, p.o., BID) | | |
| 4 | Rifaximin (102.8 mg/kg, | 4.9 ± 1.0*** | −55.7 ± 8.7 |
| | p.o., BID) + Apremilast | | |
| | (6.2 mg/kg, p.o., BID) | | |

***Significantly different from DSS Vehicle control at p < 0.001 by using one-way ANOVA followed by Dunnett t-test. [n = 6 in Water vehicle control, n = 8 in DSS vehicle control and Rifaximin (102.8 mg/kg, p.o., BID) + Apremilast (6.2 mg/kg, p.o., BID) and n = 4 in Rifaximin (102.8 mg/kg, p.o., BID) + Apremilast (2.1 mg/kg, p.o., BID)].

The rectal bleeding is one the most important feature of ulcerative colitis the scores in different treatment groups is as given in table 15.

TABLE 15
The rectal bleeding score

| | | | % change in |
|---|---|---|---|
| Sr. | | | bleeding score vs |
| No. | Treatment groups | Day-6 | DSS vehicle control |
| 1 | Water Vehicle Control | 0.0 ± 0.0 | |
| 2 | DSS (2.5%) vehicle control | 3.1 ± 0.6 | |
| 3 | Rifaximin (102.8 mg/kg, | 0.5 ± 0.5* | −84.1 ± 15.9 |
| | p.o., BID) + Apremilast | | |
| | (2.1 mg/kg, p.o., BID) | | |
| 4 | Rifaximin (102.8 mg/kg, | 0.8 ± 0.5* | −76.1 ± 16.7 |
| | p.o., BID) + Apremilast | | |
| | (6.2 mg/kg, p.o., BID) | | |

*Significantly different from DSS Vehicle control at p < 0.05 by using one-way ANOVA followed by Dunnett t-test. [n = 6 in Water vehicle control, n = 8 in DSS vehicle control and Rifaximin (102.8 mg/kg, p.o., BID) + Apremilast (6.2 mg/kg, p.o., BID) and n = 4 in Rifaximin (102.8 mg/kg, p.o., BID) + Apremilast (2.1 mg/kg, p.o., BID)].

b) The colon length is one the most important feature of ulcerative colitis the length (cm) in different treatment groups is as given in table 16.

TABLE 16
The colon length (cm)

% improvement
in colon

| | | | length vs DSS |
|---|---|---|---|
| Sr. No. | Treatment groups | Day-6 | vehicle control |
| 1 | Water Vehicle Control | 7.6 ± 0.3 | |
| 2 | DSS (2.5%) | 4.9 ± 0.2 | |
| | vehicle control | | |

| | | | |
|---|---|---|---|
| 3 | Rifaximin | 6.4 ± 0.3* | 55.1 ± 9.3 |
| | (102.8 mg/kg, p.o., | | |
| | BID) + Apremilast | | |
| | (2.1 mg/kg, p.o., BID) | | |
| 4 | Rifaximin (102.8 mg/kg, | 6.8 ± 0.4*** | 69.7 ± 13.0 |
| | p.o.., BID) + Apremilast | | |
| | (6.2 mg/kg, p.o., BID) | | |

*at p < 0.05 and
***at p < 0.001 significantly different from DSS Vehicle control by using one-way ANOVA followed by Dunnett t-test. [n = 6 in Water vehicle control, n = 8 in DSS vehicle control and Rifaximin (102.8 mg/kg, p.o., BID) + Apremilast (6.2 mg/kg, p.o., BID) and n = 4 in Rifaximin (102.8 mg/kg, p.o., BID) + Apremilast (2.1 mg/kg, p.o., BID)].

c) Histology of colon was performed to assess the grade of inflammation using Hematoxylin and eosin staining (H & E staining). The colon inflammation score evaluated by H & E staining is given in table 17 and the representative feature of ulcerative colitis and the grades in different treatment groups is as given FIG. 1.

TABLE 17
The inflammation score (H&E staining) in colon tissue

% change in
Inflammation
score vs DSS
vehicle

| Sr. No. | Treatment groups | Day-6 | control |
|---|---|---|---|
| 1 | Water Vehicle Control | 0.0 ± 0.0 | |
| 2 | DSS (2.5%) | 3.8 ± 0.3 | |
| | vehicle control | | |

| | | | |
|---|---|---|---|
| 3 | Rifaximin | 3.0 ± 0.0 | −20.0 ± 0.0 |
| | (102.8 mg/kg, p.o., | | |
| | BID) + Apremilast | | |
| | (2.1 mg/kg, p.o., BID) | | |
| 4 | Rifaximin | 1.3 ± 0.5** | −66.7 ± 12.8 |
| | (102.8 mg/kg, p.o., | | |
| | BID) + Apremilast | | |
| | (6.2 mg/kg, p.o., BID) | | |

**Significantly different from DSS Vehicle control at p < 0.01 by using one-way ANOVA followed by Dunnett t-test. [n = 3 in Water vehicle control, n = 4 in DSS vehicle control and Rifaximin (102.8 mg/kg, p.o., BID) + Apremilast (6.2 mg/kg, p.o., BID) and n = 2 in Rifaximin (102.8 mg/kg, p.o., BID) + Apremilast (2.1 mg/kg, p.o., BID)].

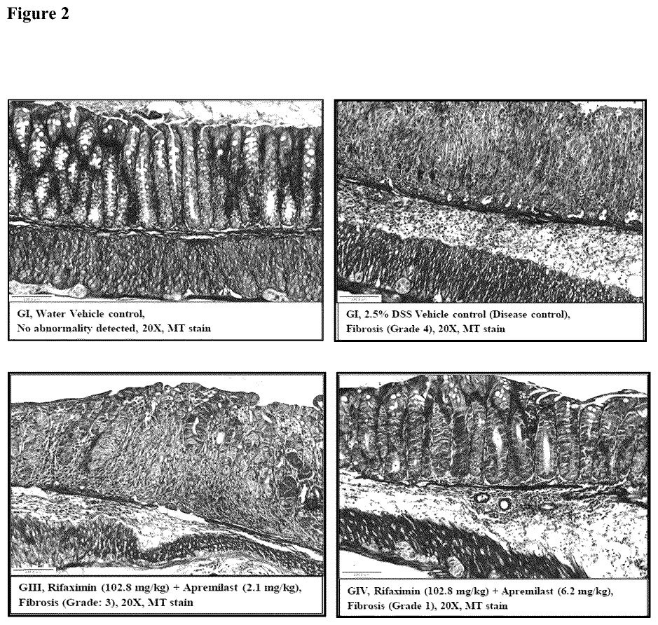

a) Histology of colon was performed to assess the grade of fibrosis by Masson's trichrome staining. The colon tissue collagen score (indicative of fibrosis) is given in table 18 and the representative histology images and grades in different treatment groups are as given FIG. 2.

TABLE 18
The collagen score (Masson's trichrome staining) in colon tissue

% change in
collagen
score
vs DSS
vehicle

| Sr. No. | Treatment groups | Day-6 | control |
|---|---|---|---|
| 1 | Water Vehicle Control | 0.0 ± 0.0 | |
| 2 | DSS (2.5%) | 3.5 ± 0.3 | |
| | vehicle control | | |

| | | | |
|---|---|---|---|
| 3 | Rifaximin | 2.5 ± 0.5 | −28.6 ± 14.3 |
| | (102.8 mg/kg, p.o., | | |
| | BID) + Apremilast | | |
| | (2.1 mg/kg, p.o., BID) | | |
| 4 | Rifaximin | 1.3 ± 0.5** | −64.3 ± 13.7 |
| | (102.8 mg/kg, p.o., | | |
| | BID) + Apremilast | | |
| | (6.2 mg/kg, p.o., BID) | | |

**Significantly different from DSS Vehicle control at p < 0.01 by using one-way ANOVA followed by Dunnett t-test. [n = 3 in Water vehicle control, n = 4 in DSS vehicle control and Rifaximin (102.8 mg/kg, p.o., BID) + Apremilast (6.2 mg/kg, p.o., BID) and n = 2 in Rifaximin (102.8 mg/kg, p.o., BID) + Apremilast (2.1 mg/kg, p.o., BID)].

### Observation:

Combination of the two different doses of Apremilast (equivalent to 10 mg and 30 mg of human dose) with Rifaximin (equivalent to 500 mg of human dose), showed significant improvement in DAI score by 36% and 56%, in rectal bleeding score by 84% and 76% and in colon length by 55% and 70% respectively. In colon histology inflammation score improvement was 20% and 67% whereas the fibrosis score improvement was 29% and 64% respectively for Apremilast 10 and 30 mg dose equivalent. In PSC-IBD model the Apremilast equivalent to 30 mg of human dose with Rifaximin (equivalent to 500 mg of human dose), showed better efficacy as compared to Apremilast equivalent to 10 of human dose in combination with Rifaximin (equivalent to 500 mg of human dose).

### Conclusion

In Mdr2KO mice model which is model for human PSC-IBD, both the combination of Apremilast (equivalent to 10 mg and 30 mg of human dose) with Rifaximin (equivalent to 500 mg of human dose) showed improvement in DSS induced ulcerative colitis model of inflammatory bowel disease. But combination of Apremilast (equivalent to 30 mg of human dose) with Rifaximin (equivalent to 500 mg of human dose) found to be more effective in PSC mediated IBD.

### Example 10

Evaluation of ADD ON Effect of FDC with Mesalamine in DSS Induced Ulcerative Colitis in C57 Mice Model

To study the ADD ON effect of FDC (combination of Apremilast equivalent to 15 mg and Rifaximin equivalent to 500 mg of human dose) with mesalamine equivalent to 800 mg of human dose in animal model, which is DSS (Dextran sulfate sodium) induced ulcerative colitis in C57 mice (Br J Pharmacol. 2019 July; 176 (13): 2209-2226. doi: 10.1111/bph.14667. Epub 2019 May 17). The colitis induced by DSS, presents good reproducibility and also mimics clinical symptoms, inflammatory markers and histopathological features which is similar to IBD in humans (Cell Mol Gastroenterol Hepatol. 2015 Mar. 1; 1 (2): 154-170. doi: 10.1016/j.jcmgh.2015.01.006., Arq Gastroenterol. 2014 April-June; 51 (2): 107-12. doi: 10.1590/s0004-28032014000200007. PMID: 25003261).

### Methods

C57 mice of 10-12 weeks age were used for this study. On the day of study initiation (day-0) animals were randomized based on their body weight in to different treatment groups as below:

TABLE 19
Treatment groups and dose levels

| Sr. | | | No. of |
|---|---|---|---|
| no. | Treatment group | Dose and route | animals |
| 1 | Water Vehicle Control | 0 mg/kg, p.o. | 06 |
| 2 | DSS (2.5%) vehicle control | 0 mg/kg, p.o. | 10 |
| 3 | Mesalamine (164.4 mg/kg, p.o., | 164.4 mg/kg, p.o., BID | 10 |
| | BID) | (Equivalent human dose = 800 mg) | |
| 4 | FDC + Mesalamine (164.4 mg/kg, | FDC [Rifaximin 102.8 mg/kg, | 10 |
| | p.o., BID) | p.o., BID | |
| | | (Equivalent human dose = 500 mg) + | |
| | | Apremilast 3.1 mg/kg, p.o., BID | |
| | | (Equivalent human dose = 15 mg)] + | |
| | | 164.4 mg/kg, p.o., BID | |
| | | (Equivalent human dose = 800 mg) | |

On day-0 animals were randomized based on body weight and from the same day onwards animals were administered with 2.5% DSS in RO water along with the test compounds till day-6.

The test compounds were formulated in Tween 80 and 0.5% Na. CMC in (0.5:99.50%) ratio and oral administration was done twice a day for six days. During the duration of the experiment, a disease activity index (DAI) score was assessed to evaluate the clinical progression of ulcerative colitis. The DAI was calculated by grading on a scale of 0-5 using the following parameters: loss of body weight (0=normal; 1=0-5%; 2=6-10%; 3=11-15%; 4=16-20%, 5=>20%), stool consistency (0=normal; 1=soft stools; 2=loose stools; 3=watery diarrhea) and rectal bleeding (0=none; 2=presence of hemoccult; 4=severe bleeding). (Ref: Cytokine 2014, 66, 30-39).

On the day of termination (Day-6), DAI score was assessed followed by nonfasted blood collection. Then animals were sacrificed, colon length was measured and then collected in 10% formalin for histological analysis or snap frozen in liquid nitrogen for other assays.

With the help of MS Excel, percent change vs disease control (DSS vehicle control) in various parameters were calculated and statistical analysis was performed using graph pad prism software. Percentage improvement of colon length was calculated by calculating the decrease in colon length due to disease from water vehicle control group and then improvement in colon length against DSS vehicle control group by considering the colon length of water vehicle control group as 100%.

### Results

a) The disease activity index (DAI) score was assessed on day-6 to evaluate the clinical progression of ulcerative colitis as given in Table 20.

TABLE 20
The disease activity index (DAI) score

% change in
DAI vs
DSS vehicle

| Sr. No. | Treatment groups | Day-6 | control |
|---|---|---|---|
| 1 | Water Vehicle Control | 0.7 ± 0.3 | |
| 2 | DSS (2.5%) | 5.2 ± 0.6 | |
| | vehicle control | | |

| | | | |
|---|---|---|---|
| 3 | Mesalamine | 3.1 ± 0.3** | −40.2 ± 5.0 |
| | (164.4 mg/kg, p.o., BID) | | |
| 4 | FDC + Mesalamine | 1.4 ± 0.6**** ^{$} | −72.2 ± 11.2 |
| | (164.4 mg/kg, p.o., BID) | | |

**at p < 0.01 and
****at p < 0.0001 Significantly different from DSS Vehicle control by using one-way ANOVA followed by Dunnett t-test.
^{$} Significantly different from Mesalamine treatment group at p < 0.05 by using t-test. [n = 6 in Water vehicle control, n = 10 in DSS vehicle control, n = 9 in Mesalamine (164.4 mg/kg, p.o., BID), n = 9 in FDC + Mesalamine (164.4 mg/kg, p.o., BID)].

b) The colon length is one the most important feature of ulcerative colitis the length in different treatment groups is as given in table 21.

TABLE 21
The Colon length (cm)

% improvement
in colon
length vs DSS

| Sr. No. | Treatment groups | Day-6 | vehicle control |
|---|---|---|---|
| 1 | Water Vehicle Control | 7.2 ± 0.2 | |
| 2 | DSS (2.5%) | 5.7 ± 0.2 | |
| | vehicle control | | |

| | | | |
|---|---|---|---|
| 3 | Mesalamine | 6.3 ± 0.2 | 39.3 ± 11.0 |
| | (164.4 mg/kg, p.o., BID) | | |
| 4 | FDC + Mesalamine | 6.7 ± 0.2* | 68.7 ± 14.8 |
| | (164.4 mg/kg, p.o., BID) | | |

*Significantly different from DSS Vehicle control at p < 0.05 by using one-way ANOVA followed by Dunnett t-test. [n = 6 in Water vehicle control, n = 10 in DSS vehicle control, n = 9 in Mesalamine (164.4 mg/kg, p.o., BID), n = 9 in FDC + Mesalamine (164.4 mg/kg, p.o., BID)].

### Observation:

Mesalamine (equivalent to 800 mg of human dose) showed significant improvement in disease activity index (DAI) score by 40%. But when we added our FDC combination (combination of Apremilast equivalent to 15 mg and Rifaximin equivalent to 500 mg of human dose) with Mesalamine (equivalent to 800 mg of human dose) it showed statistically significant 72% improvement in DAI score. When we evaluate the effect on colon length, FDC combination add on to has shown statistically significant of 69% improvement.

### Conclusion

Combination of FDC along with Mesalamine showed significantly greater improvement in DAI score (which is the most prominent clinical disease parameters of evaluation in IBD) and in colon length also the efficacy was better in Combination of FDC along with Mesalamine treatment group. It shows that our FDC add on to mesalamine (SOC) will have better efficacy as compared to mesalamine alone.

1. The pharmaceutical combination comprising,
A first component (1) Apremilast or pharmaceutically acceptable salts thereof
A second component (2) Rifaximin or isomers or pharmaceutically acceptable salts.

2. The pharmaceutical combination claimed in claim 1, wherein Apremilast is in stereoisomerically pure form.

3. The pharmaceutical combination claimed in claim 1, wherein Rifaximin is in polymorphic form.

4. The polymorph of Rifaximin as claimed in claim 3, further comprised α, β, γ, δ, ε or Z form of Rifaximin.

5. The pharmaceutical combination claimed in claim 1, wherein combination comprises a therapeutically effective amount of Apremilast and a therapeutically effective amount of Rifaximin, where the therapeutically effective amount of Apremilast is selected from 1.0 mg to 100.00 mg and the therapeutically effective amount of Rifaximin selected from 1.0 mg to 550.00 mg.

6. The pharmaceutical combination claimed in claim 1, wherein the combination comprises a therapeutically effective amount of Apremilast and the required therapeutically effective amount of Apremilast, is for administration to a human subject, and is selected from the range 10.0 mg to 30.00 mg.

7. The pharmaceutical combination claimed in claim 1, wherein the combination comprises a therapeutically effective amount of Rifaximin and the required therapeutically effective amount of Rifaximin, is for administration to a human subject, and selected from the range 400.0 mg to 500.00 mg.

8. The pharmaceutical combination claimed in claim 1, wherein the said combination is suitable for oral, parenteral or topical route of administration and the said combination is formulated to give to the patient in need thereof by once a day, twice a day (BID) or thrice a day.

9. A pharmaceutical composition comprising Apremilast, Rifaximin and suitable pharmaceutically acceptable excipients selected from a suitable disintegrant, suitable coating agent, suitable enteric coating material, a suitable filler, a suitable binder, a suitable glidant, a suitable lubricant and a suitable plasticizer.

10. The pharmaceutical composition as claimed in claim 9, wherein a suitable disintegrant is selected from cross-linked polymers: cross-linked polyvinylpyrrolidone (cross-povidone), cross-linked sodium carboxymethyl cellulose (croscarmellose sodium), L-HPC (Low substitute hydroxypropylcellulose) and/or mixtures thereof: suitable coating agent is selected from methyl acrylate-methacrylic acid copolymers, cellulose acetate phthalate (CAP), cellulose acetate succinate, hydroxypropyl methyl cellulose phthalate, hydroxypropyl methyl cellulose acetate succinate (hypromellose acetate succinate), polyvinyl acetate phthalate (PVAP), methyl methacrylate-methacrylic acid copolymers and/or mixtures thereof: suitable enteric coating material is selected from methyl acrylate-methacrylic acid copolymers, cellulose acetate phthalate (CAP), cellulose acetate succinate, hydroxypropyl methyl cellulose phthalate, hydroxypropyl methyl cellulose acetate succinate (hypromellose acetate succinate), polyvinyl acetate phthalate (PVAP), methyl methacrylate-methacrylic acid copolymers and/or mixtures thereof: suitable a filler is selected from dibasic calcium phosphate, lactose, dextrose, fructose, kaolin, sucrose, mannitol, microcrystalline cellulose, powdered cellulose, precipitated calcium carbonate, sorbitol, starch and/or mixtures thereof: a suitable binder is selected from, polyvinylpyrrolidone, hydroxypropylcellulose, hydroxypropylmethylcellulose, hydroxyethylcellulose, methylcellulose, sodium carboxymethyl cellulose, calcium carboxymethylcellulose, calcium carboxymethyl cellulose and/or mixtures thereof: a suitable glidant is selected from silica gel or colloidal silicon dioxide, talc, magnesium carbonate and/or mixtures thereof: a suitable lubricant is selected from stearic acid, a metal salt of stearic acid such as magnesium stearate, talc, colloidal silica, a wax variety such as beads wax, spermaceti, boric acid, adipic acid, sodium sulphate, fumaric acid, stearyl sodium fumarate, sucrose aliphatic acid ester, a lauryl sulphate such as sodium lauryl sulphate, magnesium lauryl sulphate, starch derivative such as corn starch, potato starch, glyceryl behenate, behenoyl polyoxyl glyceride and/or mixtures thereof and a suitable plasticizer is selected from sorbitol, glycerol, triethylcitrate, polysorbate, carnauba wax, PEG (polyethylene glycol) and/or mixtures thereof.

11. The pharmaceutical composition comprising: Apremilast, Rifaximin, suitable binder and optionally with other pharmaceutically acceptable ingredients/excipients.

12. The pharmaceutical composition as claimed in claim 11, wherein the composition is formulated for immediate release of Apremilast and Rifaximin.

13. The pharmaceutical composition as claimed in claim 11, wherein the composition apprises other pharmaceutically acceptable ingredients/excipients selected from a suitable extra granular agent, a suitable filler, a suitable glidant, a suitable lubricant and a suitable plasticizer.

14. The pharmaceutical composition according to claim 11, wherein, suitable binder is hydroxy propyl methyl cellulose: suitable filler is microcrystalline cellulose; suitable glidant is colloidal silicon dioxide; suitable lubricant is magnesium stearate; suitable extra granular agent is Hydroxy Propyl Methyl Cellulose, L-HPC and suitable plasticizer is polyethylene glycol.

15. A pharmaceutical composition comprising: Apremilast, Rifaximin, suitable disintegrant, suitable coating agent and optionally with other pharmaceutically acceptable ingredients/excipients.

16. The pharmaceutical composition as claimed in claim 15, wherein the formulation is formulated for delay release of Apremilast and Rifaximin.

17. The pharmaceutical composition as claimed in claim 15, wherein the composition comprises other pharmaceutically acceptable ingredients/excipients selected from a suitable filler, a suitable binder, a suitable glidant, a suitable lubricant and suitable plasticizer.

18. The pharmaceutical composition according to claim 15, wherein, suitable disintegrant is hydroxypropyl cellulose: suitable coating agent is methacrylic Acid copolymer: suitable filler is microcrystalline cellulose; suitable binder is: suitable glidant is colloidal silicon dioxide: suitable lubricant is magnesium stearate and suitable plasticizer is polyethylene glycol.

19. A pharmaceutical composition comprising: Apremilast, Rifaximin, a suitable enteric coating agent and optionally other pharmaceutically acceptable ingredients/excipients.

20. (canceled)

21. The pharmaceutical composition as claimed in claim 19, wherein other pharmaceutically acceptable ingredients/excipients is selected from suitable filler, suitable binder, suitable glidant, suitable lubricant and suitable plasticizer.

22. The pharmaceutical composition according to claim 19, wherein, suitable enteric coating agent is methacrylic Acid copolymer: suitable filler is microcrystalline cellulose; suitable binder is; suitable glidant is colloidal silicon dioxide; suitable lubricant is magnesium stearate and suitable plasticizer is polyethylene glycol.

23-24. (canceled)

25. The pharmaceutical composition of claim 17, where the other pharmaceutically acceptable ingredients are selected from mesalamine, suitable corticosteroids, suitable immuno-suppressants and biologics therapy.

26. A pharmaceutical kit comprising two parts divided with a perforation or a punch or other suitable means to place two medicaments separately wherein medicaments are selected from Apremilast and Rifaximin.

27. The pharmaceutical kit as claimed in claim 26, wherein the required therapeutically effective amount of Apremilast, is for administration to a human subject, selected from the range 10.0 mg to 30.00 mg.

28. The pharmaceutical kit as claimed in claim 26, wherein the required therapeutically effective amount of Rifaximin, is for administration to a human subject, selected from the range 400.0 mg to 500.00 mg.
